(19) United States
(12) Patent Application Publication
(10) Pub. No.: US 2026/0153514 A1
(43) Pub. Date: Jun. 4, 2026

(54) A METHOD FOR ANALYSING A MEMBRANE PROTEIN

(71) Applicants: Oxford University Innovation Limited, Oxford (GB); Thermo Fisher Scientific Inc., Waltham, MA (US); OMASS Therapeutics, Oxford (GB)

(72) Inventors: Carol ROBINSON, Oxford (GB); Corinne LUTOMSKI, Oxford (GB); Tarick EL-BABA, Oxford (GB); Idlir LIKO, Oxford (GB); John SYKA, Waltham, MA (US); Josh HINKLE, Waltham, MA (US)

(21) Appl. No.: 19/123,599

(22) PCT Filed: Oct. 27, 2023
(86) PCT No.: PCT/GB2023/052813

§ 371 Date: Apr. 23, 2025

(30) Foreign Application Priority Data
Oct. 28, 2022 (GB) 2215982.6

(51) Int. Cl. G01N 27/624

(52) U.S. Cl. CPC G01N 33/6848; G01N 27/628

(57) ABSTRACT

The disclosure concerns a method of analysing a sample comprising an intact membrane protein, in a sample comprising non-synthetic biological membrane material. The method involves irradiating a gas phase proto membrane protein ion with infrared irradiation from a laser source.

### FIELD OF THE INVENTION

The invention relates to a method of analysing a sample by mass spectrometry comprising an intact membrane or membrane associated protein, or a collection of these proteins, in a sample comprising non-synthetic biological membrane material.

### BACKGROUND TO THE INVENTION

Membrane proteins are involved with a wide range of cellular processes such as cell-cell adhesion, signal transduction, and solute flux. It is well-established that membrane protein structure and function can be regulated by binding to other molecules such as extracellular ligands, lipids within the membrane, and cytosolic proteins. Native mass spectrometry (nMS) provides a powerful means to study the relationship between these binding events, as they lead to distinct changes in the composition of the protein complex that can be elucidated using mass spectrometry.

Much work has been done to understand the structure, function, and activity of model systems such as isolated proteins and idealised model membrane proteins. For instance, to study membrane protein-ligand interactions by nMS, membrane proteins or complexes are typically solubilized in a membrane mimetic prior to purification. Herein, the term “protein” (for instance, a “membrane protein”) includes protein complexes, such as protein complexes comprising a plurality of associated polypeptide chains, or proteins forming a complex with one or more covalently and/or non-covalently bound ligands. The membrane protein (or protein complex) may be encased in a detergent micelle (or another suitable mimetic such as a lipid nanodisc), and then delivered into the gas phase via nanoelectrospray ionization. Once in the gas phase, the multitude of non-specifically bound detergent molecules must be removed to facilitate mass analysis of the membrane protein or complex under study (referred to as micelle removal or de-micellisation).

The de-micellisation step is typically carried out by sufficiently increasing the vibrational energy of the membrane protein-micelle complex using energetic collisions with neutral gas molecules to cleave the non-covalent bonds between the non-specifically bound detergent molecules and the membrane protein(s) under study. In general, ion-neutral collision-based ion dissociation methods are commonly referred to in the art as collision induced dissociation (CID), and, less frequently, as collision activated dissociation (CAD). In the art, specific forms and implementations of CID are often referred to by various other names and acronyms.

However, such CID based approaches to de-micellisation can be problematic since (i) the detergent molecules optimized for membrane solubilization are not always amenable to mass spectrometry due to their relatively high binding affinities to the proteins that inhibit their gas phase removal, (ii) detergent solubilization itself can perturb protein structure, function, and activity, and may destabilize membrane protein complexes, (iii) weakly bound ligands can be removed along with the detergent during the micelle removal step, and (iv) the vibrational excitation sufficient to induce removal of detergent molecules by CID may also sufficiently energize the target membrane protein and alter the composition of the membrane protein complex.

The need to circumvent the use of detergents in membrane protein mass spectrometry research is clear, and can in principle be circumvented by analysing membrane proteins directly from membraneous environments. However, the above enumerated limitations for de-micellisation by collision-based approaches are common to the removal of non-specifically bound membrane (bulk) lipid molecules in the gas phase from membrane protein or protein complex ions. Furthermore, removal of lipid molecules is associated with its own problems, as will be discussed below.

Membrane proteins in biological environments will be associated with cell membranes, solvents, and a plurality of other associated molecules. A membrane protein associated with a large number of molecules, such as non-specifically adducted lipids (bulk lipids), residual solvent molecules, and so on (that would generate many combinations of molecular weights), is difficult to analyse. Consider a sample containing multiple proteins, each having the same amino acid sequence with identical (covalent) post translational modifications (i.e. they are all the same proteoform) but each associated with varying combinatorial associations (n=0, 1, 2 . . . ) of non-specifically bound adducts (bulk lipids, residual solvent molecules, and so on). Each protein, carrying differing combinations of adducts, will have a different total mass. Accordingly, when subjected to analysis by mass spectrometry, such a sample will not usually yield distinct m/z peaks in the resulting mass spectrum. Instead, the highly heterogenous mixture of ion m/z corresponding to the same proteoform will manifest in a mass spectrum as a broad continuum of unresolved m/z peaks often undisguisable from noise. However, distinct peaks are desirable as they are utilized to determine protein molecular weight, through the matching of specific series of m/z peaks of mass-to-charge (m/z) ratios associated with differing degrees of ionization of a proteoform of a specific molecular weight. Such molecular weight information can provide useful insights into membrane protein structure, interaction, and function.

MS analysis of a liberated protein, a protein from which all or most all of the non-specifically associated molecules that comprises the complex cellular milieu have been removed, can yield a wealth of information. The molecular weight information provided by the mass-to-charge ratio (m/z) of the liberated intact protein or protein complex ions is not always sufficient for molecular identification. In the case where the protein under study has been enriched in the cellular environment, it can be interrogated by tandem mass spectrometry techniques including techniques involving multiple stages of m/z selection and dissociation. This allows determination of protein composition, and sub-unit structure. Further, determination of absolute molecular composition (that is, the amino acid sequence and type and location of posttranslational modifications) are potentially enabled. Such tandem mass spectrometry studies of intact proteins and protein complexes also have the potential to provide sequence tags identifying the protein or constituent proteins; the location and type of post translational modifications of a constituent protein; and information on the molecular weight and, potentially, compound class and structure of non-covalent binding partners. For instance, Skinner et al. (“Top-down characterization of endogenous protein complexes with native proteomics”, Nature Chemical Biology, Vol. 14, 2018, pp 36-43) have described the detailed and complex compositional and structural information which can be obtained for soluble proteins and complexes by analysing them in this way.

However, it is not common to glean such detailed information from membrane proteins present amid many other biomolecules, including the native membranes which they are immersed within or associated with. This is due to the difficulties in analysing a membrane protein which requires interactions with non-covalently associated lipids to maintain its stability in the solution phase. The multitude of membrane protein-lipid interactions hampers the ability to observe a discrete m/z peak for protein from a m/z spectrum, which precludes the utility of any downstream tandem mass spectrometry analyses. Thus, it is desirable to be able to analyse, by mass spectrometry, a membrane protein directly from its biological (membraneous) environment in a manner that liberates the protein of any non-specifically bound lipid molecules whilst otherwise retaining the integrity of the protein complex.

Accordingly, workers in this field have made considerable efforts to provide simplified systems which enable the analysis of proteins in their native environment. They have attempted to effect the removal of bulk lipids and residual solvent molecules by subjecting gas phase ions containing proteins to various forms of CID. Most commonly this has been implemented either in one of the intermediate pressure stages of a mass spectrometer inlet by biasing electrode potentials such that ions undergo sufficiently energetic collisions with the atmospheric gas molecules to effect CID as they transit through the mass spectrometer's inlet region, or by delivering the ions to a conventional RF multipole collision cell after transiting the inlet where the ions would undergo energetic collisions with a collision gas. Such approaches have also been used with some success to liberate membrane protein and protein complex ions from membrane mimetics such as detergents (depending upon the detergent used) and lipid nano-discs. One exemplary study in this area has been performed by Gault et al. (“Combining native and ‘omics’ mass spectrometry to identify endogenous ligands bound to membrane proteins”, Nature Methods, Vol. 17, 2020, pp 505-508).

However, the inventors have found these purely collisional approaches to liberation of protein ions for analysis to be very much less successful in the analysis of protein samples extracted in association with vesicles directly from the biological environment. That is because of difficulties in removing non-specifically bound lipids (also referred to herein as “de-lipidisation”). The inventors have found that (1) the collisional activation of the membrane protein-bulk lipid system in order de-lipidise is inefficient, causing the poor yields of fully liberated “free” protein ions, (2) the acceleration potentials used to subject vesicle ions to energetic collisions may adversely affect the transmission of the ion in the mass spectrometer, often in a m/z dependent manner, and (3) the kinetic energy threshold of collisional activation required for de-lipidisation is generally much higher than that required for de-micellisation, which limits the scope of protein specific non-covalent ligand interactions that can be preserved in the liberated protein ions to only those that are extremely strong. In other words, non-covalently bound protein specific ligands that bind to the periphery of a membrane protein are almost surely lost during the de-lipidisation, preventing the full observation, identification and characterisation of such membrane protein-ligand complexes.

To successfully perform “top down” sequence analysis of intact protein ions via tandem mass spectrometry (also referred to as MS/MS or MSⁿ), one must effectively and efficiently generate the intact protein precursor ions, m/z isolate (or m/z select) them and then convert them, ideally, into sequence determinative product ions by dissociating them. A variety of appropriate dissociation techniques are known in the art. These product ions can optionally be subjected to further manipulations such as further steps of m/z isolation (m/z selection) and charge reduction via methods which are known in the art to produce the final generation product ions. The final generation product ions are subjected to m/z analysis and detection to generate a product ion m/z spectrum. This product ion m/z spectrum then is interpreted to provide the amino acid sequence information or protein identification. For the product ion spectrum to be suitable for sequence analysis, it is necessary to dissociate a large number of precursor ions. That enables the detection of sufficient numbers of final generation product ions to generate interpretable isotopic m/z peak envelopes in the m/z spectrum, corresponding to the large number of different sequence product ions.

For instance, in the case where the product ions are 10-100 residue amino acid fragments derived from backbone cleavages along the primary structure of a high molecular weight precursor (such as a membrane protein or membrane protein complex), there are a large number of different dissociation pathways which produce a corresponding very large number of product ion types (due to very large numbers of possible bond cleavage locations). Thus, the precursor ion signal will be divided between a large number of product ion types (C- and N-terminal sequence ions, internal fragment ions, side chain loss ions etc.). Thus, unless a sufficient yield of the initial starting ion (such as a membrane protein stripped of bulk lipids and solvent) can be obtained, tandem mass spectrometric analysis of the starting ion will not succeed. Any mass spectra obtained will have such low signal-to-noise ratios that sequence ion m/z peaks in the mass spectrum cannot be discerned. Therefore, the success of any tandem mass spectrometry experiment is dictated by the quantity/flux of precursor ions that are converted to product ions.

Accordingly, due to their low yield of ions of liberated proteins, the inventors have found that conventional collision-based techniques to effect de-lipidisation are generally not suitable to provide enough liberated intact membrane protein ions of any type to perform any sort of tandem mass spectrometry experiments.

Assuming a sufficient quantity of the liberated protein precursor ions can be produced, many fragmentation methods are available to induce non-covalent and covalent bond cleavage to generate product ions suitable for the analysis of the composition of the precursor protein. For instance, along with various forms of CID, ion-electron and ion-ion methods such as electron capture dissociation (ECD) and electron transfer dissociation (ETD) have been used for dissociation of intact proteins. Other known fragmentation techniques utilize light from a laser source to induce bond cleavages by irradiating precursor ions with either ultraviolet (UV) or infrared (IR) light to trigger dissociative electronic or vibrational transitions, respectively. These methods are commonly referred to in the art as ultraviolet photodissociation (UVPD) and infrared multi-photon photodissocation (IRMPD). All of these techniques have the capacity to break bonds along the peptide backbone of protein ions to generate amino acid fragment ions that are suitable for compositional analysis of a precursor. For example, IRMPD has been used to analyse the amino acid sequences of purified intact soluble proteins (see for instance, Li, Nguyen, Ogorzalek Loo, Campuzano, & Loo “An integrated native mass spectrometry and top-down proteomics method that connects sequence to structure and function of macromolecular complexes” Nat. Chem. vol. 12, 2018, pp 139-148). IRMPD has also been used to break peptide bonds and fragment underlying enzymatically produced peptides (see, for instance, Crowe & Brodbelt “Infrared multiphoton dissociation (IRMPD) and collisionally activated dissociation of peptides in a quadrupole ion trap with selective IRMPD of phosphopeptides”, J. Am. Soc. Mass Spectrom., vol. 15, 2004, pp 1581-1592).

Accordingly, the inventors have recognised that in view of the foregoing there is a need to provide a charge-independent method to selectively remove non-specifically bound molecules such as the bulk membrane lipids from membrane proteins and their complexes. Such a method is needed to produce intact liberated membrane protein ions in high yield which would then render them amenable to top-down analysis by tandem mass spectrometric methods.

There is further a need for alternative and/or improved methods for characterising intact membrane proteins in native or native-like environments. For example, there is a need for methods to characterise intact membrane proteins without relying on (or in the absence of) detergents and other non-natural surfactants. As referenced above, whilst detergents can assist in solubilisation of membrane proteins they typically perturb native protein structure and may destabilise native protein complexes, such that characterisation or analysis data obtained from such samples may not accurately reflect the underlying native protein or protein complex. The presence of non-natural detergent or surfactant molecules can further hamper accurate data collection as data obtained may be convoluted by the presence of residual detergent or surfactant molecules in the sample, which may be particularly problematic for synthetic detergent molecules which may bind relatively strongly to the membrane protein. Accordingly, data obtained may be characteristic of an adduct of the native protein or protein complex and the detergent. Such data may be time consuming or complex to deconvolute. Conversely, attempts to remove the detergent may disrupt the structure of the underlying native protein or protein complex.

### SUMMARY OF THE INVENTION

The inventors have discovered that in vacuo infrared (IR) activation of gas phase ions comprising membrane proteins within lipid membrane fragments can be highly effective in stripping away the bulk lipid molecules and residual solvent molecules, to provide liberated gas phase intact membrane protein ions. Further, the inventors have found that bulk lipid molecules can be preferentially removed from gas phase proto membrane protein ions while allowing the liberated gas phase intact membrane protein ions to retain many of their other non-covalent binding partners (including e.g., peptides, drugs and other small molecules specific to the function of the protein). In test cases, medium resolution m/z analysis of such liberated gas phase intact membrane protein ions produced m/z spectra with discrete representative m/z peaks that had assignable average m/z values. The m/z peaks corresponding to such IR activation liberated protein ions in obtained m/z spectra were found to have significantly superior signal-to-noise ratios (and intensities) compared to the m/z peaks obtained achievable in when the instrument was configured to utilize known collisional activation approaches.

Without wishing to be bound by theory, it is speculated that these superior results may be observed for the following reasons.

It is possible that the infrared wavelength photons used in the accompanying examples are more readily absorbed by the bulk lipid molecules than by the proteins, as their wavelengths excite particular vibrational modes of bonds within the bulk lipids. Accordingly, the infrared wavelength may be chosen such that it is on-resonance or sufficiently near resonance with vibrational modes of certain non-specifically adducted molecules (such as bulk lipid molecules).

The method of the invention involves initially generating a gas phase proto membrane protein ion from a sample. In a typical experiment, many such gas phase proto membrane protein ions are produced. The gas phase proto membrane protein ions are confined (for instance in an ion trap). The confined gas phase proto membrane protein ions are exposed to infrared (IR) radiation from a laser source. However, in addition to being exposed to the infrared radiation, the confined ions are typically subject to high numbers of near thermal energy collisions with gasses such as helium, argon, air, nitrogen, or hydrogen (among others). These collisions have a cooling effect on trapped ions which counteracts the heating caused by absorption of the infrared laser radiation. It is believed that this combined effect quickly establishes an equilibrium level of vibrational activation of the confined gas phase proto membrane protein ions. Hence the IR-driven de-lipidisation process may be controlled not only by adjusting the duration and intensity of the infrared beam transmitted through the trapped ion cloud, but also by the choice of gas and the gas pressure and therefore its collisional cooling effect.

The small molecule protein specific binding partners of membrane proteins may likely have somewhat higher binding affinities to the protein than the non-specifically bound solvent and bulk lipid molecules. Thus, it is possible that these combined aspects may provide a fine of control of the of the process. Such control may enable parameters for IR activation to be selected such that the gas phase proto membrane protein ions can be heated sufficiently precisely to “evaporate” the relatively weaker non-covalently bound solvent and bulk lipid molecules while maintaining the non-covalent interactions that bind the membrane protein internally and to its other, protein specific, small molecule binding partners (thus producing liberated gas phase intact membrane protein ions, typically consisting of the protein and binding partners specific to the protein).

Further, though lipid molecules have a significantly smaller mass and correspondingly fewer vibrational degrees of freedom than proteins, the inventors believe that membrane lipids have a higher density of bonds that absorb relatively more efficiently in the IR (and particularly the wavelength band of the IR laser light that has been used in the accompanying examples). Thus, the lipid molecules are perhaps heated to a greater extent by the infrared radiation than the protein that they partially encapsulate. If such differential heating occurs it would be expected to promote more efficient liberation of the protein ions from membrane bulk lipids.

The method described herein is particularly suited to the analysis of membrane proteins contained in non-synthetic biological membrane material, as it is capable of selectively removing the complex mixture of membrane bulk lipid molecules attached to the membrane protein while substantially maintaining the composition of the underlying membrane protein (complex) intact.

Accordingly, the invention provides a method of analysing a sample comprising an intact membrane protein, the method comprising:
(i) providing a sample comprising a non-synthetic biological membrane material comprising the intact membrane protein, a solvent, and bulk lipid molecules;
(ii) ionising the sample to produce a gas phase proto membrane protein ion, said gas phase proto membrane protein ion comprising the intact membrane protein non-specifically bound to one or more bulk lipid molecules and to one or more residual solvent molecules;
(iii) confining the gas phase proto membrane protein ion;
(iv) detaching the one or more residual solvent molecules and one or more non-specifically bound bulk lipid molecules from the intact membrane protein by irradiating the confined gas phase proto membrane protein ion with infrared radiation from a laser source to produce a gas phase intact membrane protein ion; and
(v) m/z analysing and detecting the gas phase intact membrane protein ion and/or an ion derived therefrom.

The m/z analysis may precede m/z detection, or they may be concomitant.

Step (iv) may be referred to as “de-lipidation”, or “the de-lipidation step”.

Particularly preferred examples of a non-synthetic biological membrane material include a biological membrane material extracted from or secreted directly by a cell, an organelle, a viral envelope or a vesicle.

A key advantage of the present method is that it is able to fully liberate a membrane protein from residual solvent and bulk lipid molecules. This can be done with higher probability than the CID based methods of the prior art, and thus can generate from a population of proto membrane protein ions the gas phase intact membrane protein ions in higher quantities. Thus, during analysis where many bulk lipidated protein ions are subjected to the method of the invention, there is usually a higher yield of fully liberated protein ions relative to the CID de-lipidation method. In a preferred aspect of the method, the gas phase intact membrane protein ion is free of non-specifically bound bulk lipid molecules. Similarly, the gas phase intact membrane protein ion is preferably free of residual solvent molecules. The gas phase intact membrane protein ion may further be free of other, non-lipid, non-specifically bound molecules.

A related advantage of the present method is that it permits the characterisation of intact membrane proteins in native or native-like environments, such as in the substantial or complete absence of non-naturally occurring detergents and other non-natural surfactants. Whilst the use of detergents is encompassed within the present invention, it is an advantage of the present methods that samples which are at least substantially free of detergent can be assessed. In particular, it was previously understood that endogenous membrane proteins could not be readily liberated from vesicles derived from their native lipid bilayer for characterisation by mass spectrometry without the use of detergents and/or other non-natural surfactants, with the disadvantages of incorporating such substances as discussed above. The ability to release and probe intact membrane proteins without the requirement for detergents is a surprising advantage of the present invention.

A significant advantage of the method of the invention is that it provides a higher yield of ions which can be subjected to further analysis. Since the gas phase intact membrane protein ion is produced by the method in sufficient abundance, it can be manipulated and analysed as one would any other native protein (such as a protein complex). In particular, these gas phase intact membrane protein ions can be subjected to analysis by tandem mass spectrometry methods. For instance, the gas phase intact membrane protein ions may be subjected to an ion transformative process to produce one or more product ions, and the one or more product ions may be detected, and m/z analysed (optionally after being subjected to a further transformative process, such as charge reduction). The first generation product ion itself may be subjected to m/z selection prior to a further ion transformative process, and so on. Accordingly, step (v) may comprise
(va) subjecting the gas phase intact membrane protein ion to an ion transformative process to produce a first generation product ion; and
(vb) m/z selecting and optionally detecting the first generation product ion.

The method may further comprise:
(vc) subjecting the first generation product ion to a further ion transformative process to produce a second generation product ion; and
(vd) m/z selecting the second generation product ion, and optionally m/z analysing and detecting the second generation product ion.

Each ion transformative process may produce one or more product ions. Accordingly, in practice, subjecting the gas phase intact membrane protein ion to an ion transformative process may produce one or more product ions. Where a plurality of such ions are produced, each ion is a first generation product ion. Where a plurality of such first generation product ions are produced, one or more of them may be m/z selected (and optionally detected) in step (vb). Each first generation product ion produced may be the same or different. If step (vc) is performed, step (vc) may be performed on one or more of the first generation product ions. Similarly, in practice, subjecting a first-generation product ion to an ion transformative process may produce one or more (second generation) product ions. Where a plurality of such ions are produced, each ion is a second generation product ion. Each second generation product ion may be the same or different. Where a plurality of such second generation ions are produced, one or more of them may be m/z selected (and optionally m/z analysed and detected) in step (vd).

For instance, in a “top down” tandem MS experiment, a particular charge state of the liberated ions of a membrane protein are m/z selected (for instance, all other ions of m/z outside a relatively narrow band of m/z corresponding to the particular charge state of the protein may be ejected from the relevant part of the apparatus) and then subjected to further activation in order to dissociate the covalent bonds of the protein to produce ions which, upon analysis, are diagnostic of the amino acid sequence(s) of the intact protein.

A wide variety of options are available for the ion transformative process step(s), also referred to herein as activation step(s) or ion transformation step(s). These methods include infrared multiphoton dissociation, electron transfer dissociation, activated ion electron transfer dissociation, electron capture dissociation, UV photodissociation, and collision-induced dissociation (CID). Particular examples of CID include collision cell type collisional activation and RF ion trap type resonant collisional activation.

Preferred among these ion transformative processes is infrared multiphoton dissociation, IRMPD, as it is typically more reliable and efficient in effecting dissociation of liberated large membrane protein and protein complex ions. It is characteristic of such liberated ions, which have been initially generated as proto membrane protein ions via nESI (nanoelectrospray ionisation), that they are highly charged (typically having charge states, z, of 10 or more), and that they have relatively low charge densities. Thus, their mass-to-charge ratios typically exceed 3,000 Dalton/elementary charge (Thompson, Th) and are typically in the m/z range of 4,000 Th to 12,000 Th. Protein and protein complex ions of such mass and charge are not generally efficiently dissociated into sequence information product ions by electron-based methods such as ETD. Further, because of the very large mass difference between collision gas molecules (which typically have a mass of 40 Da or less) and the liberated protein or protein complex precursor ions (which generally have a mass greater than 25 kDa, frequently in the range of 50 kDa to 150 kDa, and even extending above 200 kDa), the most common methods and implementations for collision induced dissociation, collision cell type CID and ion trap type resonant CID, can also often fail to impart sufficient vibrational excitation to the liberated precursor ions so as to yield a rich range of fragment ions resulting from the cleavage of protein backbone that, upon m/z analysis and detection, yield m/z spectra that can be utilised for sequence analysis.

In contrast, the absorption of IR photons into protein ions appears, as would be expected, to be approximately proportional to molecular weight and approximately matches how the number of vibrational states in a protein increases with molecular weight. Further, it is expected that the extraction of vibrational energy from the ions though collisions at near thermal kinetic energies with collision gas, would be proportional to the surface area or cross-section of the ions which in turn would be expected to scale at something less than in proportion to molecular weight. So the net heating (effective temperature) of the proto membrane protein ions and intact membrane protein ions due to exposure of ions to the IR photons would not be expected to be strongly dependent upon ion molecular weight. Indeed, the inventors have not observed a strong molecular weight dependence on the IR laser output power required to induce IRMPD of the nESI-produced membrane protein ions (comprising intact proteins and protein complexes) they have studied, and these proteins ions have ranged in molecular weight from about 30 kDa to well above 150 kDa. The inventors have also observed that IR activation can induce non-covalent bond cleavage releasing small molecule binding partners either as ions or neutrals from the gas phase intact membrane protein ion. Further, in some instances, IR activation can interrupt the non-covalent interactions that bind the proteins in multi-protein complex ion to produce ions that are observed as m/z peaks in the associated tandem m/z spectra and that correspond to the individual protein sub-units or various incomplete combinations of the protein subunits.

IR activation at moderately higher irradiation levels than would be suitable to effect removal of bulk lipids from the nESI-generated protein and protein complex containing ions (proto membrane protein ions) will generally also effect cleavage of both protein backbone (typically the dominant dissociation pathway) and side chain covalent bonds. Variation of the activating IR photon flux to which the membrane protein ions are exposed (generally controlled by adjusting the laser output power) as well as the duration of that exposure provides control of the extent of the dissociation. At a fixed photon flux level sufficient to induce cleavage on the covalent bonds on the backbone of a protein, extending the IR photon exposure time produces successive generations of product ions since, absent measures to remove first generation IRMPD product ions from further exposure to IR photons, each generation of product ions is subjected to continued IR radiation and activation leading to further dissociation as the exposure time is extended. The final product ion population will include a mixture of N-terminal and C-terminal-containing sequence ions and a large number of other internal fragment ions. By internal fragment ions is meant fragment ions which are product ions that contain neither the N-terminus nor the C-terminus of the protein.

The number of possible internal fragments scales with the number, N, of amino acid residues in the protein approximately as (N−1)²/2 and the number of possible C- and N-terminus ions (sequence ions) scales as 2(N−1). An individual gas phase intact membrane protein ion would be expected to produce as product ions a pair of sequence ions and some variable number of internal fragment ions. The number of internal fragment ions generated per gas phase intact membrane protein ion will increase with longer activation times. Similarly, with longer activation times the masses of the pair of sequence ion that will be produced will decrease (fewer amino acid residues in length). Each successive dissociation cleaves the original gas phase intact membrane protein ion into more and smaller product ions. While there is some amino acid specificity to where cleavage will occur due to vibrational activation, this evolution in the composition of the types of product ions (also referred to as fragment ions, in this context) means that the abundance of individual sequence ion types produced from an initial population of gas phase intact membrane protein ions is generally greater than any one particular type of internal fragment ion produced.

Methods to inhibit production of multiple generations of photodissociation product ions are known in the art and have been referred to as product ion parking or product ion protection. Such methods could be incorporated into the methods of the invention to improve the final abundance of product ions (final generation ions) of higher molecular weight.

Without methods to improve abundance of product ions of high molecular weight, these can be difficult to detect above the electronic noise baseline signal. However, these large and highly charged product ions are suitable for m/z analysis and detection by charge detection mass spectrometry (CDMS) methods which are known in the art. In particular, CDMS methods such as those described by J. O. Kafader and colleagues [Kafader, Jared O., et al. “Measurement of individual ions sharply increases the resolution of orbitrap mass spectra of proteins.” Analytical chemistry 91.4 (2019): 2776-2783, and Kafader, Jared O., et al. “Multiplexed mass spectrometry of individual ions improves measurement of proteoforms and their complexes.” Nature methods 17.4 (2020): 391-394.] would be advantageous for obtaining mass spectra and tandem mass spectra of the liberated membrane protein and protein complexes by methods according to the invention described herein. Accordingly, step (v) may comprise detecting and m/z analysing the gas phase intact membrane protein ion and/or an ion derived therefrom by a CDMS method.

A further key advantage of the method is the ability to optimise the rate of energy supplied to the gas phase proto membrane protein ion, in order to control the process whereby residual solvent molecules and non-specifically bound bulk lipid molecules are detached. With the use of an infrared laser, the period of irradiation, the output power level of the laser, the wavelength, the photon beam profile and (if a pulsed laser is used rather than a continuous wave laser) the pulse width of the laser can all be easily adjusted. Thus, the irradiation conditions can be adjusted to selectively deposit enough energy to break non-covalent bonds between proteins and non-specifically bound molecules. Accordingly, the process may comprise determining the irradiation parameters to be used in step (iii) by repeatedly performing the process and gradually increasing the energy of the laser radiation and/or the time period for which the irradiation of the gas phase proto membrane protein ion is performed until the gas phase intact membrane protein ion can be detected after step (iv).

Of course, in any subsequent IRMPD fragmentation step, these conditions can also be adjusted to produce an observable series of N- and C-terminal sequence ions as well as (sequence) interpretable series of internal fragment ions.

The invention will be described in more detail hereafter.

### BRIEF DESCRIPTION OF THE FIGURES

FIG. 1 is a schematic diagram of an exemplary apparatus used in performing the exemplary method of the invention.

FIG. 2 contains representative series of mass-to-charge ratio (m/z) spectra depicting the use of the method of the invention to release protein ions from non-specifically bound lipids from E coli membrane fragments containing, among other membrane proteins and complexes, MacAB. Symbols label m/z peaks corresponding to charge state series corresponding common molecular weights. Values in the top-right of each spectrum are the intensity values for the most intense peak. Asterisks (*) are used to label m/z peaks that do not collectively correspond to a sequence of charge states of a protein or other high molecular weight compound. The infrared irradiation time was held fixed at 10 ms. IR Laser power output setting (nominally % of full output-uncalibrated) is indicated for each spectrum. The figure illustrates the effect of laser intensity in generating gas phase intact membrane protein ions from a biological matrix derived from lipid membrane fragments containing intact membrane protein ions. The figure shows that optimised parameters suitable for liberating membrane proteins and complexes can identified through locating the set of parameters that produces, among the highest yield of liberated ions, the highest diversity of m/z peaks corresponding to gas phase intact membrane and membrane associated proteins, as well as m/z peaks that are signatures of ligand binding and/or post translational modification (PTM) retention.

FIG. 3 illustrates representative m/z spectra depicting the use of collisional activation (50 V, 100 V, 150 V, 200 V, and 250 V accelerating potentials) in the ion inlet optics (also often referred to in the art as in-source CID) to release membrane protein and membrane protein complex ions from non-specifically bound lipid vesicles (membrane fragments) containing, among other molecules, the membrane protein MacAB. Values in the top-right of each spectrum are the intensity values for the most intense peak. Asterisks (*) are used to label m/z peaks that do not correspond to any continuous charge state series and thus probably do not correspond to protein-derived ions.

FIG. 4 contains a comparison of protein m/z peaks obtained via the liberation of, among other membrane proteins, MacAB from E coli membrane vesicles (membrane fragments) at the optimized collisional activation voltage (Inlet CID using 100 V accelerating potential) and IR irradiation (5% of full output-uncalibrated-laser intensity for 10 ms). Poor signal intensities are observed for the peaks corresponding to the 102,775 Da protein released by in-source CID. When utilizing infrared irradiation, m/z peak intensities corresponding to the MW 102,755 Da protein (in this case a protein complex) are clearly observed at high intensities. In addition, a series of satellite m/z peaks at higher m/z values that are signatures of ligand binding and/or PTM retention are observed. The molecular weight of this satellite distribution of m/z peaks is consistent with a ligation or modification of 129 Da.

FIG. 5 illustrates the optimisation of IR parameters for liberating intact membrane protein ions of, among other membrane proteins (including complexes), the BamABCDE complex, from lipid vesicles ions. The figure contains MS spectra obtained using three IR laser power settings (7%, 10%, and 15% of full output-uncalibrated) that demonstrate the release intact protein ion of, among other membrane proteins, the intact BamABCDE complex from ionized vesicles (proto membrane protein ions) formed from E. coli outer membranes. IR laser irradiation time was held fixed at 10 ms.

FIG. 6 illustrates m/z spectra collected at optimised IR irradiation parameters to liberate the BamABCDE complex, along with other proteins, and subsequent IRMPD (covalent bond fragmentation) following m/z isolation of an m/z peak consistent with the intact BamABCDE complex to produce product ions for sequence identification. FIG. 6(A) (top spectrum) is a mass spectrum from ionization of E. coli lipid vesicles containing, among other membrane proteins, the BamABCDE complex and generated by liberating intact membrane protein ions of the said membrane protein complex using the optimised IR irradiation parameters (determined by the experiment depicted in FIG. 5). In FIG. 6(A) (bottom spectrum), the intact membrane protein ions corresponding to the m/z peaks indicated by the asterisk in the top spectrum (corresponding to the 28+ charge state of BamABCDE) were isolated and subjected to ion activation by high quantities of IR photons (i.e., IRMPD) to induce peptide bond cleavage; the new distribution of product ions was then m/z analysed (MS²). FIG. 6(B) contains a detailed view of two m/z ranges in FIG. 6A, bottom spectrum (the MS² spectrum). These were inspected for the presence of m/z peaks indicative of sequence fragment product ions from the BamE subunit (i.e., the b₂₀ and y₂₇). The bolded text above the m/z peaks in each detailed view denote the monoisotopic m/z peaks corresponding to b₂₀ and y₂₇ fragment ions (top and bottom, respectively). In FIG. 6(C), the sequence information on the left shows the sequence of the BamE subunit indicating the stretches of amino acids that the b₂₀ and y₂₇ fragment ions correspond to. The table on the right shows the experimental and theoretical molecular weights of these fragment ions, and demonstrates they are well within acceptable thresholds for sequence identification (i.e., <15 ppm).

FIG. 7 contains an exemplary mass spectrum illustrating the use of IR irradiation to release intact membrane protein ions of membrane proteins/complexes having MW 39 kDa, 148 kDa, and 271 kDa, as well as other molecules, from proto membrane protein ions that are ionized naturally secreted extracellular vesicles (exosomes) from human fluid.

FIG. 8 shows the impact of IR laser output power on the liberation of membrane proteins from a native lipid bilayer for detection by mass spectrometry. FIG. 8(A) was obtained with a low laser output power of 3% (corresponding to 1.8 W) and an irradiation time of 25 ms. Only soluble proteins are observed in the mass spectrum. Adjacent charge state series are denoted by circles. FIG. 8(B) was obtained with a high laser output power of 9% (corresponding to 5.4 W) and the same irradiation time of 25 ms. Rhodopsin (a 7 transmembrane protein) has been liberated from the native lipid bilayer and represents the major charge state distribution in the mass spectrum (denoted by stars).

FIG. 9 demonstrates the use of the method of the invention in the sequencing of rhodopsin following its liberation from the lipid bilayer. A single charge state for rhodopsin was isolated and subjected to infrared multiphoton dissociation (15% (9 W) for 5 ms irradiation time) to induce protein backbone cleavages, producing the tandem mass spectrometry (MS²) spectrum in panel (A). b/y-type fragment ions were matched using spectral matching to a predicted MS² spectrum. Regions of interest corresponding to “proteoforms”—protein forms that are modified from the canonical sequence via the presence of post-translational modifications are shown: (B-C) glycosylation, (D) phosphorylation, and (E) palmitoylation. FIG. 9(F) is a map of the entire repertoire of fragment ions that were matched to those predicted for the unmodified and modified forms of rhodopsin. Approximately 14% sequence coverage was obtained, providing complete confidence in the assignment of the peaks in the mass spectrum (FIG. 8) to rhodopsin.

### DETAILED DESCRIPTION OF THE INVENTION

The present invention relates to a method for analysis of intact membrane proteins by mass spectrometry by which intact membrane proteins can be transferred into the gas phase as ions directly from their native, membraneous environment; bulk lipid and residual solvent molecules can be removed, and the gas phase intact membrane protein ions can then be m/z analysed and detected or, through further transformative processes (such as fragmentation or charge reduction) may be converted into other ions derived therefrom prior to m/z analysis and detection. Specifically, the invention provides a method of analysing a sample comprising an intact membrane protein, the method comprising:
(i) providing a sample comprising a non-synthetic biological membrane material comprising the intact membrane protein, a solvent, and bulk lipid molecules;
(ii) ionising the sample to produce a gas phase proto membrane protein ion, said gas phase proto membrane protein ion comprising the intact membrane protein non-specifically bound to one or more bulk lipid molecules and to one or more residual solvent molecules;
(iii) confining the gas phase proto membrane protein ion;
(iv) detaching the one or more residual solvent molecules and one or more non-specifically bound bulk lipid molecules from the intact membrane protein by irradiating the confined gas phase proto membrane protein ion with infrared radiation from a laser source to produce a gas phase intact membrane protein ion; and
(v) m/z analysing and detecting the gas phase intact membrane protein ion and/or an ion derived therefrom.

### The Sample

The method of the invention concerns the analysis of a sample comprising a non-synthetic biological membrane material. The non-synthetic biological membrane material comprises, at least, an intact membrane protein; a solvent; and bulk lipid molecules. The sample is generally a liquid sample.

The sample preferably comprises the intact membrane protein in the condition in which it is generally found in its natural biological environment. That is, the sample preferably contains the intact membrane protein in the membrane environment in which it is found in nature. Thus, the intact membrane protein is preferably in a “wild” state.

The non-synthetic biological membrane material may alternatively be referred to as a naturally-occurring biological membrane material. That is, the non-synthetic biological membrane material is a species which is obtained or obtainable from a biological cell or virus. Examples of biological membrane materials are membrane materials obtained or obtainable from a plant or animal cell (preferably an animal cell, particularly preferably a human cell); a bacterial cell; or a virus. Accordingly, the non-synthetic biological membrane material differs from synthetic membranes and synthetic membrane mimetics, such as detergent micelles, lipid nanodiscs and synthetic lipid monolayers or bilayers. Like a non-synthetic biological membrane material, a synthetic membrane material will typically comprise an intact membrane protein and a solvent; it will also contain bulk lipid molecules and/or detergent molecules. However, in a synthetic membrane, the bulk lipid molecules and/or detergent molecules are artificially assembled in vitro to form a synthetic monolayer or bilayer. The non-synthetic biological membrane material described herein excludes synthetic membrane materials such as synthetic membrane mimetics.

Typically, the non-synthetic biological membrane material comprises a membrane (and membrane protein) which is harvested or liberated from a cell (including a bacterial cell) or a virus. That is, the non-synthetic-biological membrane material is typically obtained or obtainable from a cell or a virus.

Where the non-synthetic biological membrane material is obtained or obtainable from a cell, the cell may be a cultured cell or a harvested cell (that is, harvested from an organism). In such cases, the non-synthetic biological membrane material may comprise the cell membrane of the cell. For instance, the non-synthetic biological membrane material may be obtainable by lysing the outer plasma membrane of the cell. The outer plasma membrane comprises the membrane (generally a lipid bilayer) that defines the boundaries of the cell, together with membrane protein(s) embedded therein. The aforementioned outer plasma membrane may be extracted directly after lysis to yield a non-synthetic biological membrane material.

The non-synthetic biological membrane materials obtainable from a cell are not limited to the outer plasma membrane. Liberated organelles or vesicles can be extracted and yield a non-synthetic biological membrane material.

An organelle is generally defined as a specialised sub-unit from within a cell, having a specialised function within the cell. Examples of organelles include but are not limited to mitochondria, and the endoplasmic reticulum, and examples of vesicles include multivesicular bodies, endosomes, and phagosomes. Organelles can be liberated (and thus comprise liberated protein) from cells by lysing the cell and then extracting the organelle.

A vesicle is generally defined as a fluid-filled sac surrounded by a membrane. Vesicles can be present within a cell or can be found external to a cell; they are often secreted from the surface of cells. Examples of vesicles found external to cells include but are not limited to exosomes and extracellular vesicles. Vesicles external to cells can be harvested (that is, liberated, and so may contain liberated protein) directly from biological fluids or aerosols such as blood and saliva. Vesicles internal to cells are obtained by lysing the surrounding cell and then isolating the vesicles.

Vesicles and organelles comprise a membrane (generally a lipid bilayer) at their external edge, and the membrane generally has one or more membrane proteins embedded therein.

A non-synthetic biological membrane material can be obtained from a vesicle or organelle by lysing the vesicle or organelle.

Thus, in a preferred embodiment, the non-synthetic biological membrane material is a material which is extracted from or secreted directly by a cell, an organelle, a viral envelope or a vesicle. For instance, the non-synthetic biological membrane material may be obtained or obtainable by lysing an external membrane of a cell, an organelle or a vesicle, or from a viral envelope, and isolating the said membrane. Preferably, the non-synthetic biological membrane material is obtained or obtainable by lysing an external membrane of a vesicle, and isolating the said membrane.

The non-synthetic biological membrane material described herein may more concisely be referred to as a non-synthetic membrane material, or a biological membrane material.

The bulk lipid molecules are the lipid molecules present in the membrane (such as an external membrane of a cell, vesicle or organelle, or a viral envelope) from which the non-synthetic biological membrane material is obtained. Consequently, at least some of the bulk lipid molecules are generally arranged in the form of a membrane. For instance, some or all of the bulk lipid molecules may be arranged as a lipid monolayer or a lipid bilayer. In some cases (for instance where the non-synthetic biological membrane material is obtained from a multilamellar vesicle), some or all of the bulk lipid molecules may be arranged in the form of a plurality of bilayers.

In the sample provided in step (i) of the method, the non-synthetic biological membrane material may comprise an intact vesicle, or an intact virus or viral lipid envelope. For instance, the bulk lipid molecules may form a lipid bilayer or bilayers in which the intact membrane protein is present, and wherein the bilayer or bilayers form an intact unilamellar or multilamellar vesicle. Where such an intact vesicle is present, it is typically submicron-sized. By “submicron-sized” is meant that the largest diameter of the vesicle is less than 1 μm.

More typically, though, the sample generally does not comprise an intact cell, vesicle, organelle or viral envelope. Rather, the sample containing the non-synthetic biological membrane material is generally obtained by lysing the cell, organelle, vesicle or viral envelope and extracting membrane material therefrom. In addition, the extracted membrane material may have been subjected to further processes in order to provide the sample, such as sonication, which may disrupt membrane structures such as lipid monolayers or bilayers originally present in the cell/vesicle/organelle/viral envelope. Thus, although the non-synthetic biological membrane material comprises bulk lipid molecules originating from a membrane, the entirety of the membrane structure may not be maintained. Rather, the sample typically comprises a membrane fragment (such as a fragment of a lipid bilayer) associated with the intact membrane protein. In particular, the sample may comprise a submicron-sized membrane fragment associated with the intact membrane protein. By submicron-sized is meant that largest dimension of the fragment is less than 1 μm.

Some of the bulk lipid molecules are bound directly to the intact membrane protein. The bulk lipid molecules are said to be non-specifically bound. By this is meant that the lipid molecules do not modulate the structure, function, assembly or activity of the membrane protein. Thus, the lipid molecules are bound to the intact membrane protein at a site or at sites not responsible for conferring or modulating the structure, function, assembly, or activity of the membrane protein. For instance, the lipid molecules are not bound at a receptor site of the protein. The bulk lipid molecules which are bound to the intact membrane protein are bound by non-covalent interactions with the protein. The majority of the bulk lipid molecules will not be bound directly to the protein but will be bound to other bulk lipid molecules, typically within a monolayer or bilayer arrangement.

Accordingly, the non-synthetic biological membrane generally comprises an intact membrane protein which is present in or attached to a membrane comprising the bulk lipid molecules. For instance, the non-synthetic biological membrane typically comprises an intact membrane protein which is non-specifically bound to a lipid bilayer or a lipid monolayer (preferably a lipid bilayer), wherein the lipid bilayer comprises the bulk lipid molecules. The lipid bilayer may form a vesicle, but is more typically a small fragment of a lipid bilayer (generally sub-micron sized).

The bulk lipid molecules may be of one type or multiple different types. The bulk lipid molecules are generally those which are typically found in non-synthetic biological membranes (also referred to as native membranes). Thus, the bulk lipid molecules generally include glycerophospholipids. The bulk lipid molecules may also include phosphatidyl ethanoloamine, phosphatidyl glycerol, cardiolipins, phosphatidylcholines, phosphatidylserine, cholesterol, diacylglycerols, fatty acids, long-chain fatty acids, glycosphingolipids, sphingomelins, and lipopolysaccharides.

By an intact membrane protein is meant that the protein exists in its native state, as in its native membrane environment. That is, any specifically-bound covalent binding partners bound to the membrane protein in its native state remain attached to the membrane protein. Intact means that the protein is not fragmented. For instance, where the membrane protein comprises multiple sub-units or monomers, the intact membrane protein typically comprises all of these sub-units in their native arrangements, rather than one or more fragments thereof. However, minor changes to the native arrangement can be permissible. For instance, a subcomplex of a membrane protein which is a macromolecular protein assembly is regarded as “intact”, or “native” even if some of the sub-units are not assembled as in the biologically active state.

Membrane proteins can be grouped into integral membrane proteins and peripheral membrane proteins. Integral membrane proteins may have one or more amino acid segments embedded within a membrane and may be non-covalently bound to the bulk lipid molecules of the membrane. Peripheral membrane proteins may be temporarily associated with the bulk lipid molecules and/or integral membrane proteins. In an embodiment, the intact membrane protein is an integral membrane protein. In another embodiment, the intact membrane protein is a peripheral membrane protein.

Membrane proteins may be composed of one (mono) or more (multi) associated polypeptide chains. Thus, the intact membrane protein may be a monomeric or a multimeric membrane protein, for example an oligomeric membrane protein. Oligomeric membrane proteins include both homooligomeric (identical polypeptide chains) and heterooligomeric (different polypeptide chains) proteins. Accordingly, in an embodiment, the intact membrane protein comprises two or more non-covalently associated monomers, which monomers may be the same or different.

The intact membrane protein may have a molecular weight of from about 10³ Daltons to about 10¹² Daltons, for instance from about 5×10³ Daltons to about 10⁶ Daltons.

A key advantage of the present method is that it can be used to decipher the structure and/or chemical composition of an unknown protein. Accordingly, the structure and/or chemical composition of the intact membrane protein may be unknown.

Non-limiting examples of classes of membrane protein include G protein-coupled receptors (GPCRs such as opsins, e.g. rhodopsin), membrane transporters, membrane channels, ATP-binding cassette transporters (ABC-transporters), proton driven transporters, solute carriers, outer membrane proteins (OMPs), ATP synthases, and protein and ligand translocases. Specific examples of membrane proteins include anchor cell fusion failure protein 1 (AFF-1), p-barrel assembly machinery (BAM), bacterial molecular chaperone DnaK, cytochrome bo3, the CydAB cytochrome bd oxidase complex, energy-transducing Ton complex, multidrug efflux pumps such as AcrABZ-TolC and MdtABCTolC, and ATP synthase, membrane protein complexes in the respiratory chain {e.g. complexes 1-V), adenine nucleotide translocase 1 (ANT-1), and subunits thereof.

The intact membrane protein may be in the form of a complex with a ligand. Indeed, an advantage of the present method is that the structural information which can be derived concerning the intact membrane protein may include the stoichiometry in which one or more ligand(s) bind to the membrane protein, the binding location of the ligand(s), and information on conformation changes that occur upon binding of a ligand to the membrane protein.

Binding of the ligand to the intact membrane protein may be via a non-covalent or a covalent interaction. It will typically be via a non-covalent interaction. In particular, binding of the ligand to the intact membrane protein may be via intermolecular forces such as ionic bonds, hydrogen bonds and van der Waals forces. Binding of the ligand to the intact membrane protein may be reversible or irreversible. In an embodiment, the ligand is bound to the intact membrane protein via a reversible bond. Generally, the ligand is bound to the intact membrane protein with some degree of specificity.

Examples of ligands include, but are not limited to, an RNA strand; a metabolite; a drug (which may be a therapeutic agent or a diagnostic agent); a metal cofactor; a lipid, a nucleotide; and a nucleoside. Preferred examples of ligands include an RNA strand, a metabolite, a drug, a metal cofactor and a lipid.

Where the ligand is a lipid, the lipid differs from the bulk lipid molecules as it is part of the protein (which is, in this context, a complex) and not the membrane. Examples of lipids which may be ligands include fatty acids, glycerolipids, glycerophospholipids, sphingolipids, sterol lipids, prenol lipids, saccharolipids and polyketides.

Where the intact membrane protein is in the form of a complex with a ligand, the complex may comprise one or more such complexed ligands. Each such ligand may independently be selected from the exemplary ligands described above, including an RNA strand; a metabolite; a drug (which may be a therapeutic agent or a diagnostic agent); a metal cofactor; a lipid, a nucleotide; and a nucleoside.

The intact membrane protein may comprise one or more post-translational modifications. Some forms of post-translational modification may include the formation of a complex including the membrane protein and one or more ligands. Other forms of post-translational modification may include the addition or modification of a functional group within the protein. Particular examples of post-translational modifications include glycosylation and phosphorylation.

The sample further comprises a solvent. The solvent is typically water, but other solvents may be present (for instance, alcohols such as ethanol or methanol). More than one solvent may be present.

The sample may comprise components in addition to those described above.

For instance, the sample may additionally comprise a mass-spectrometry compatible buffer. Preferably, the mass-spectrometry compatible buffer is one which maintains the structural integrity of the intact membrane protein. In particular, the mass-spectrometry compatible buffer is one which can maintain the structural integrity of the intact membrane protein when said protein is associated with a submicron-scale membrane fragment or vesicle, and solvent molecules. Suitable mass-spectrometry compatible buffers can be readily selected by those skilled in the art. They include ammonium buffers, such as ammonium acetate or ammonium bicarbonate. Ammonium acetate is particularly preferred.

Where the sample comprises a mass spectrometry compatible buffer, said buffer is typically present at a concentration of at least 150 mM, e.g. 250 to 1000 mM, e.g. 400 to 600 mM.

The sample may alternatively or additionally comprise one or more of the species described above as ligands. Often, these species can be added to the sample in order to generate in intact membrane protein wherein one or more ligands form a complex with the membrane protein. Accordingly, the sample may comprise an RNA strand; a metabolite; a drug (which may be a therapeutic agent or a diagnostic agent); a metal cofactor; a lipid, a nucleotide; and a nucleoside. Where such a ligand is added to the sample in order to promote the formation of a complex comprising the ligand, the ligand is generally present in a molar excess compared to the membrane protein. For instance, the ligand may be present in the sample at a molar ratio of 2:1 or more compared to the membrane protein.

In addition, the sample may comprise more than one type of intact membrane protein. For instance, the sample may comprise 2, 3, 4 or more different intact membrane proteins. The intact membrane proteins may comprise the same underlying membrane protein complexed with a plurality of different ligands. Alternatively or additionally, a plurality of different membrane proteins may be present in the sample.

The pH of the sample is typically a native pH. A native pH is a pH typically found in a biological cell environment. Thus, the pH of the same is typically from pH 5 to pH 8.

Typically, the membrane protein will be present in the (optionally sonicated) solution in an amount of at least 0.5 mg/mL, e.g. from 0.5 to 20 mg/mL, e.g. from 1 to 10 mg/mL.

The sample is preferably largely free of detergent. For instance, the sample may comprise detergent at a concentration of less than 100 μM, e.g. less than 1 μM. Preferably, the solution is substantially free of detergent or entirely free of detergent. The sample may be at least substantially free of detergent. The sample or solution may be at least substantially free of non-naturally occurring surfactant molecules. Non-natural detergents include DDM (n-dodecyl-β-D-maltoside), n-nonyl-β-D-glucopyranoside (NG) and octyltetraglycol (C₈E₄). Accordingly, in some embodiments the sample or solution may be at least substantially free, or entirely free, of (n-dodecyl-β-D-maltoside), n-nonyl-β-D-glucopyranoside (NG), and/or octyltetraglycol (C₈E₄). The sample or solution may comprise less than about 100 μM, e.g. less than about 50 μM, such as less than about 20 μM, e.g. less than about 10 μM, such as less than about 1 μM, e.g. less than about 0.1 μM or less than about 0.01 μM of a detergent. Similarly, the non-synthetic biological membrane material is typically free or at least substantially free of detergent.

### The Gas Phase Proto Membrane Protein Ion

The method described herein involves producing from the sample a gas phase proto membrane protein ion, and then selectively removing solvent and bulk lipid molecules from that gas phase proto membrane protein ion to produce a gas phase intact membrane protein ion.

The gas phase proto membrane protein ion comprises the intact membrane protein non-specifically bound to one or more bulk lipid molecules, and to one or more residual solvent molecules. The gas phase proto membrane protein ion is produced by ionising the sample. As explained above, the sample typically comprises an intact membrane protein associated with bulk lipid molecules in the form of a membrane (typically a lipid bilayer). Generally, the membrane is merely a submicron-sized fragment, rather than an entire vesicle or other body. Accordingly, the gas phase proto membrane protein ion typically comprises an intact membrane protein associated with bulk lipid molecules in the form of a membrane (typically a lipid bilayer, although a monolayer is also possible), and residual solvent molecules.

The proto membrane protein ion may be a charged ion which is produced directly on ionisation of the sample. More generally, though, an ion produced by ionisation of the sample will shed some non-specifically bound molecules as it is transferred through the inlet to the mass spectrometer and through various stages of vacuum to reach the location where it will be confined and subsequently irradiated. Accordingly, the proto membrane protein ion also refers an ion which is derived from the ion produced directly by ionisation of the sample by loss of one or more neutral or ionic bulk lipid molecules, and/or one or more solvent molecules.

The gas phase proto membrane protein ion may be referred to more concisely herein as “the gas phase PMP ion”, or “the initial gas phase MP ion”. Alternatively, the gas phase proto membrane protein ions may simply be referred to herein as proto membrane protein ions as once ions have entered in through the inlet of the mass spectrometer they must be understood to be in the gas phase.

The residual solvent molecules are molecules of the solvent present in the sample. Accordingly, the residual solvent molecules are typically water molecules, but may also include other solvent molecules such as alcohol molecules.

Although some lipid molecules and solvent molecules may be lost during the ionisation process, generally the gas phase PMP ion comprises a plurality of bulk lipid molecules and a plurality of residual solvent molecules.

Generally, the PMP ion comprises at least 5 bulk lipid molecules. For instance, the gas phase PMP ion may comprise 20 or more, 50 or more or 100 or more bulk lipid molecules. Of course, in practice the process produces a large distribution of gas phase PMP ions wherein the number of bulk lipid molecules varies from gas phase PMP ion to gas phase PMP ion.

Similarly, the gas phase PMP ion generally comprises at least one residual solvent molecules. For instance, the gas phase PMP ion may comprise 50 or more, 100 or more or 1000 or more residual solvent molecules. Again, in practice the process produces a large distribution of PMP ions wherein the number of residual solvent and bulk lipid molecules varies from PMP ion to PMP ion.

Typically, the gas phase PMP is at least substantially free of detergent. Typically the gas phase PMP comprises less than 1000, such as less than 100, e.g. less than 50 such as less than 20 e.g. less than 10 e.g. less than 5, 4, 3, 2 or 1 detergent molecule. In some embodiments the gas phase PMP does not comprise any detergent.

The process further comprises irradiating the gas phase PMP ion to detach the residual solvent molecules and non-specifically bound bulk ligand molecules. This process produces a gas phase intact membrane protein ion. As this involves liberating the intact membrane protein ion from residual solvent molecules and non-specifically bound bulk ligand molecules, the gas phase intact membrane protein ion may also be referred to as a “liberated intact membrane protein ion”, a “gas phase liberated intact membrane protein ion”, “a gas phase intact membrane protein ion, or an intact membrane protein ion”. More concisely, the gas phase intact membrane protein ion may be referred to as a “gas phase IMP ion” or an IMP ion.

It is generally possible, during the ionisation and irradiation processes, to remove all solvent molecules from the intact membrane protein, as the solvent molecules are small and often easily detached. Accordingly, the gas phase intact membrane protein ion does not comprise residual solvent molecules.

The irradiation process is capable of fully “evaporating” bulk lipid molecules and solvent molecules from the gas phase PMP ion. However, a key advantage of this irradiation process is that the extent of that evaporation can be finely controlled so as to limit or wholly avoid the concurrent loss other non-covalently bound protein specific ligands from the membrane protein and avoid dissociation of the protein sub structure. This means that the extent to which bulk lipid molecules are removed can be finely controlled relative the control afforded by the CID based methods of the art. The inventive method makes it possible to remove all bulk lipid molecules and all solvent molecules. The gas phase intact membrane protein ion generally consists of the intact membrane protein, including any ligand(s) complexed thereto. The gas phase intact membrane protein ion consists only of the intact membrane protein together with all of the ligand(s) complexed thereto upon creation of the initial PMP ion from which it was derived. For instance, the gas phase intact membrane protein ion may consist only of the intact membrane protein, in ionised form. That is, the gas phase intact membrane protein ion is completely liberated from bulk lipids and solvent molecules. Thus, the gas phase intact membrane protein ion is free of bulk lipid molecules and solvent molecules (i.e., it comprises 0 bulk lipid molecules and 0 solvent molecules).

Where the gas phase intact membrane protein ion is bound to one or more (protein specific) ligand(s), in said gas phase intact membrane protein ion the intact membrane protein exists in the form of a complex with one or more ligands. As explained above, a ligand, where present, is usually non-covalently bound to the membrane protein. It may be advantageous for the gas phase intact membrane protein ion to comprise a ligand, for example where it is desired to study the binding between the ligand and the membrane protein. Accordingly, the gas phase intact membrane protein ion may comprise one or more non-covalently bound ligands (the ligand(s) being bound to the membrane protein).

The ligand is as described herein. Accordingly, the ligand is preferably selected from a lipid, an RNA strand, a metabolite, a drug and a metal co-factor. Where the ligand is a lipid, the lipid differs from the bulk lipid molecules. That is, the chemical structure of the lipid differs from the chemical structure of the bulk lipid molecules.

In other embodiments, it may be intended to study the structure of the membrane protein in the absence of ligands. Accordingly, in some cases the gas phase intact membrane protein ion is free of other, non-lipid, non-specifically bound molecules. (That is, the gas phase intact membrane protein ion is free of both bulk lipid molecules and other non-specifically bound molecules). In particular, in some cases the gas phase intact membrane protein ion is free of ligands.

In the gas phase intact membrane protein ion, it is preferred that the membrane protein maintains its native conformation.

The gas phase intact membrane protein ion itself could be directly m/z analyzed and detected. Advantageously, one or more ions derived from the gas phase intact membrane protein ion are m/z analysed and detected. These downstream products of the gas phase intact membrane protein ion can be produced in a variety of different ways, as will be described in more detail below. In general, it is to be understood that a reference to an ion derived from the gas phase intact membrane protein ion refers to a product ion generated from the gas phase intact membrane protein ion. A product ion which is directly produced from the gas phase intact membrane protein ion is referred to as a first-generation product ion. A product ion which is produced from a first-generation product ion may be referred to a second-generation product ion. Second- and subsequent generation product ions may be referred to as next-generation product ions.

Thus, an ion derived from the gas phase intact membrane protein is any generation product ion produced by one or more ion transformative processes involving single or multiple ion-ion, ion-neutral, ion-surface, photon-ion, electron-ion interactions. There are many different such ion transformative processes that are known in the art. A gas phase intact membrane protein ion, or first- or next-generation product ion, may be transformed into one or more product ions by such processes. Some are applicable to cations and some are applicable to anions and some of them are applicable to both. These one or more processes may be simultaneous or sequential and may involve the production of any number, including zero, of intermediate derived product ions that are further subject to such processes.

Accordingly, an ion derived from the gas phase intact membrane protein ion may be defined as a product ion produced from the gas phase intact membrane protein by an ion transformative process.

Said ion transformative processes and said interactions may be dissociative, such as the ion-neutral collisions that are used to effect various forms of CID, the single photon-ion interactions used to effect ultraviolet photodissociation (UVPD), or the multiple photon interactions used to effect IRMPD. Said ion transformative processes may involve multiple types of interactions occurring concurrently, such as in Activated-Ion Electron Transfer Dissociation (AI-ETD) where multiply charged analyte polypeptide cations undergo dissociative electron abstractive reactions with electron transfer reagent anions while also being subject to irradiation by IR photons.

Said ion transformative processes and said interactions may be reactive and involve the making and breaking of both covalent and non-covalent chemical bonds. For example, an intact membrane protein ion or a derived ion may be subject to an ion-ion interaction with reagent ions wherein the reagent ion attaches to it by either covalent or non-covalent bonds.

Said ion transformative processes and said interactions may be charge reducing. An example of such a charge reducing interacting would be a non-dissociative ion-electron type reaction (ECnoD) where a free electron is captured by a multiply charged precursor gas phase intact membrane protein cation or cationic intermediate derived ion and the resultant charge reduced radical ion remains stable and does not dissociate. A further example of a charge reducing interaction is an ion-ion proton transfer reaction. For a gas phase intact membrane protein cation or a derived cationic ions which has been ionized through multiple protonation, reaction with a proton transfer reagent anion will cause a proton to be abstracted from the cation, without inducing dissociation, to yield a product ion that is reduced by one charge.

A product ion may in some cases be a fragment of the gas phase intact membrane protein ion. A fragment is an ion produced by dissociation of another ion (such as the gas phase intact membrane protein ion). A fragment is typically produced by dissociation of a covalent bond. A product that is the result of cleaving of one or more non-covalently bound subunits or ligands from the gas phase intact membrane protein would also be considered a fragment. The fragment may be a fragment obtained directly by dissociating the gas phase intact membrane protein ion, or by dissociating a fragmentation product of the gas phase intact membrane protein ion (e.g. by fragmenting a first- or next-generation product ion).

It is to be understood that the product ion(s) derived from the gas phase intact membrane protein ion include ions which have been charge-modified (generally through ion-ion or ion-electron reactions) to increase or decrease their charge, in order to aid m/z analysis and detection and the interpretation of the corresponding m/z spectra.

Step (i)—Provision of the Sample

A suitable process for providing a sample comprising a non-synthetic biological membrane material is described in PCT/GB2019/052421, the entirety of which is incorporated herein by reference.

In general, step (i) comprises isolating a non-synthetic biological membrane material from a biological sample. The biological sample is not particularly limited. By way of example, it may be a blood sample; a saliva sample; a bacterial culture sample; a collection of cells from, e.g., a biopsy; an entire organ comprised of multiple cell types, e.g., a brain or pancreas; or a tissue culture, e.g., cells grown outside of a living organism.

The non-synthetic biological membrane material may comprise a protein liberated directly from a biological sample. In other cases, some initial processing of the sample may be needed in order to access the membrane material of interest. For instance, where the membrane protein of interest is contained in a membrane which is internal to a cell, step (i) may involve releasing the said membrane from the cell by lysing the cell. For instance, where the non-synthetic biological membrane of interest is the membrane of an organelle or intracellular vesicle, the process may comprise providing a sample comprising a biological cell, and lysing the cell to release an organelle and/or an intracellular membrane.

Further, in a preferred embodiment, step (i) comprises rupturing a non-synthetic biological membrane to produce the non-synthetic biological membrane material. It has previously been found that rupturing a cell membrane prior to ionisation produces gas phase proto membrane protein ions which are more susceptible to analysis. Further, the inventors have previously found that this rupturing of a membrane to produce intact membrane proteins associated with membrane fragments (typically submicron-sized membrane fragments) can efficiently and conveniently be achieved by sonication. Thus, in an embodiment, step (i) comprises sonicating the sample.

Suitable sonication conditions are discussed in PCT/GB2019/052421.

The sample may be sonicated for more than 1 minute. The sample may be sonicated for less than 5 minutes. For instance, the sample may be sonicated for from 2 to 4 minutes, e.g. from 2 to 3 minutes, e.g. for 2.5 minutes.

The sample may be sonicated intermittently, e.g. by cyclically applying and removing ultrasound. For instance, ultrasound may be applied in cycles of 1 to 5 seconds ‘on’ and 3 to 10 seconds ‘off’, e.g. 2 to 4 seconds ‘on’ and 5 to 7 seconds ‘off’. When the sample is sonicated intermittently, ‘off’ periods are still taken into account when calculating the time period over which sonication is applied.

Sonication may be applied using a probe sonicator, such as a Vibra-Cell VCX-500 Watt, Sonics.

Sonication may be carried out at a temperature of lower than 20° C., e.g. at a temperature of lower than 15° C., e.g. lower than 10° C. In some embodiments, sonication may be carried out on ice. This is to stop the sample from heating up as a result of the sonication.

Accordingly, in a particularly preferred embodiment, step (i) comprises isolating a non-synthetic biological membrane from a biological sample, and rupturing the non-synthetic biological membrane to produce a non-synthetic biological membrane material. Typically, the rupturing process produces submicron-sized membrane fragments, associated with an intact membrane protein. Preferably, the rupture is performed by sonication.

If the sample does not contain a mass-spectrometry compatible buffer, step (i) typically also comprises suspending the non-synthetic biological membrane material in aqueous solution comprising a mass-spectrometry compatible buffer. This step may be performed before or after any sonication step, if it is performed. Mass-spectrometry compatible buffers include ammonium buffers, such as ammonium acetate or ammonium bicarbonate. Ammonium acetate is particularly preferred.

Further, the aqueous solution comprising a mass-spectrometry compatible buffer typically also has an ionic strength similar to that of physiological conditions, and a pH of approximately 5 to 8. These conditions are believed to best preserve the endogenous (native) confirmation of the intact membrane protein, including its non-covalent interactions with any ligands, through the process of ionisation into the gas phase.

In practice, the sample generally contains a plurality of membrane proteins. The sample may contain multiple chemically identical membrane proteins (that is, proteins having an identical amino acid sequence, identical post-translational modifications, and so on). Typically, the sample contains a plurality of differing membrane proteins. The membrane proteins present may or may not have identical amino acid sequences. Where the sample contains multiple membrane proteins having the same amino acid sequence, the membrane proteins present may nonetheless differ from one another chemically (for instance by the number and/or location of post-translational modifications). Thus, the sample typically contains a plurality of membrane proteins having differing chemical structures. For instance, the sample may comprise two or more membrane proteins, each having a differing chemical structure.

Step (ii)—Ionisation

In step (ii), the sample of step (i) is ionised to produce a proto membrane protein ion, which is as defined above. The ionisation process is generally performed by some form of electrospray ionisation, preferably by nanoelectrospray ionisation or by static electrospray ionisation. Any ionization method that has the capacity to produce gas phase proto membrane protein ions (in accordance with the definition above) would be suitable to perform the invention.

In a nanoelectrospray ionisation process, nano-sized droplets are produced. This can be achieved by routine adjustment of the electrospray conditions, for example, using electrospray capillary emitters (particularly those that have been drawn to a tip with a diameter of less than 10 μm, preferably <5μ, most preferably between 0.5-2 μm).

In a preferred embodiment, the ionisation process of step (ii) is a native electrospray ionisation process. In a native electrospray ionisation process, the conditions during the electrospray procedure are kept close to physiological conditions. In particular, the pH of the sample during the electrospray process is maintained at a physiological pH. The pH is typically from 5 to 8. In such conditions, the native conformation of the intact membrane protein is optimally preserved.

Electrospray ionization is usually performed at or near atmospheric pressure. The resultant gas phase proto membrane protein (PMP) ions are as described herein, and comprise bulk lipid molecules (in some cases, even entire vesicles), together with an imbedded intact membrane protein. The intact membrane protein (in the sample and in the gas phase PMP ion and the gas phase intact membrane protein ion) may be in the form of a complex, for instance with one or more ligands. The gas phase PMP ion also comprises residual solvent molecules.

There is usually some degree of de-solvation and detachment of the bulk lipid molecules just subsequent to the ionisation process, due to the transport of the gas phase PMP ions produced by the electrospray process from the near atmospheric pressure region where they were generated into the vacuum of the mass spectrometer.

Subsequent to ionisation the proto membrane protein ions are transported into the vacuum of the mass spectrometer by passing the sample through a heated capillary tube or a suitably small orifice into a region at a pressure much below atmospheric pressure. For static electrospray ionisation and nanoelectrospray ionization the emitter tip is located within a few millimetres of the capillary entrance or orifice. For instance, the process may involve passing the proto membrane protein ion through a capillary tube heated to a temperature of greater than 250° C., for instance from 250 to 450° C., e.g. from 300 to 400° C. These high temperatures assist with the removal of residual solvent molecules without denaturing the intact membrane protein.

A mass spectrometer with an appropriately configured nano electrospray ion source may be used to perform the ionisation step. A suitable example is a Thermo Scientific™ Orbitrap Eclipse™ Tribrid™ mass spectrometer with Thermo Scientific™ NanoSpray Flex NG™ ion source configured for static native electrospray ionisation. This instrument utilizes a heated metal capillary atmosphere to vacuum interface. A modified version of that instrument with that ion source and ion source configuration has been used to perform the exemplary methods described herein.

In practice, as noted above, the sample generally contains a plurality of membrane proteins. Accordingly, ionising the sample as in step (ii) typically produces a plurality of gas phase proto membrane protein ions (also referred to as a plurality of gas phase PMP ions or a population of gas phase PMP ions). Each gas phase proto membrane protein ion is as described herein.

Each gas phase proto membrane protein ion comprises an intact membrane protein. The intact membrane protein present in each of the gas phase PMP ions within a plurality of gas phase PMP ions may be the same or different. In some cases, each gas phase PMP ion within a plurality of gas phase PMP ions comprises an intact membrane protein having an identical chemical structure. Alternatively, a plurality of gas phase PMP ions may comprise intact membrane proteins with differing chemical structures. Each gas phase PMP ion within the plurality of gas phase PMP ions may comprise an intact membrane protein having a different chemical structure. For instance, a plurality of gas phase proto membrane protein ions may comprise two or more gas phase proto membrane protein ions, wherein each gas phase proto membrane protein ion comprises an intact membrane protein having a differing chemical structure.

Upon ionisation of the sample, the produced plurality of gas phase PMP ions will have a distribution of charge states. Each gas phase PMP ion within the said plurality may be ionized to any one of a range of charge states (even where each gas phase PMP ion is comprised of an intact membrane protein that has the same amino acid sequence, or is chemically identical). For instance, the plurality of gas phase PMP ions may comprise two or more gas phase PMP ions, wherein each gas phase PMP ion has differing charge state.

Further, each gas phase PMP ion comprises one or more bulk lipid molecules and one or more residual solvent molecules. The number of bulk lipid molecules present in each gas phase PMP ion within the plurality of gas phase PMP ions may be the same or different. Similarly, the number of residual solvent molecules present in each gas phase PMP ion within the plurality of gas phase PMP ions may be the same or different. Generally, for a large plurality of gas phase PMP ions, it will include gas phase PMP ions with a broad distribution of numbers of bulk lipid molecules.

The ionisation step produces a gas phase PMP ion as described above. However, this is typically not itself suitable for m/z analysis or m/z selection for tandem m/z spectrometry experiments (m/z being the mass to charge ratio expressed as mass, m, per number of elementary charges, z). That is because, in practice, the population of gas phase PMP ions typically produced in step (ii) generally has a very broad m/z distribution as each gas phase PMP ion in the population of gas phase PMP ions includes variable numbers of bulk lipid molecules as well as variable numbers of residual solvent molecules. Moreover, since within even a plurality of gas phase PMP ions including individual gas phase PMP ions comprising identical membrane proteins and types and numbers of bulk lipid and solvent molecules (identical chemical composition), the gas phase PMP ions will have a range of charge states. Thus, the distribution of generated gas phase PMP ions would appear in an m/z spectrum only as continuity of unresolved ion signal producing a somewhat elevated baseline, essentially indistinguishable from noise. To produce a m/z spectrum with discrete representative m/z peaks that would have assignable average m/z values, a substantial fraction of the gas phase PMP ions would need to have all of all of their bulk lipid molecules and residual solvent molecules removed.

Step (ii) transfers the membrane protein (in the form of a PMP ion) into the gas phase. Accordingly, the PMP ion and all ions derived thereon (gas phase intact membrane protein ion, product ions, and so on) are all gas phase ions. Such ions may be taken to be in the gas phase even where no explicit reference to the gas phase is made.

Step (iii)—Confinement

Once the gas phase PMP ion is produced and transported into the vacuum of the mass spectrometer, it is then confined. By “confined” it is meant that the ion is localised within an electromagnetic field. This localisation may be around a point, or along an axis or some curvilinear path. Confinement indicates bounding of the ion motion and location in either two or three dimensions. A gas phase proto membrane protein ion which has been confined may be referred to as a “confined gas phase proto membrane protein ion”, the gas phase PMP ion itself being defined as above.

The gas phase proto membrane protein ion, and the gas phase intact membrane protein it is converted into in step (iv), are confined (that is, bounded in its motion in at least two dimensions in an electromagnetic field) throughout step (iv) and generally at least a portion of step (v) of the method. If one or more further steps are performed after step (iv), any ion(s) produced therein may also be confined during those steps.

In practice, a plurality of gas phase proto membrane protein ions are generally confined in step (iii). The plurality of gas phase proto membrane protein ions are as defined above. The population of gas phase PMP ions is typically confined, or localised, within a volume of space. The localised population of gas phase PMP ions may be referred to as an ion cloud. Accordingly, the following optional method steps may similarly be performed on a plurality of gas phase intact membrane protein ions produced in step (iv). Alternatively or additionally, such processes may be performed on a plurality of ions derived from the gas phase intact membrane protein ion or on a plurality of gas phase intact membrane protein ions produced in step (iv).

Any suitable apparatus may be used to confine the gas phase proto membrane protein ion (and/or the gas phase intact membrane protein ion and/or an ion derived therefrom). Suitable examples of apparatus which may be used to confine these species include a mass filter (such as a quadrupole mass filter); a radio frequency ion guide (such as a radio-frequency two dimensional multipole ion guide, a radio-frequency stacked ring ion guide, or a radio frequency stacked ring ion funnel), or an ion trap (a typical example being a dual-pressure radio frequency quadrupole linear ion trap). One example is a radio frequency multipole ion guide (ion routing multipole) which can be operated as an ion guide or an ion trap by adjusting the magnitudes of the radio frequency (RF) and direct current (DC) potentials applied to its electrodes.

The confinement of a proto membrane protein ion, an intact membrane protein ion, or an ion derived from a membrane protein ion may be performed using one such apparatus, or a plurality of apparatus. More than one piece of such apparatus may be used to confine an ion of any such type during each of steps (iii), (iv) and (v).

For instance, exemplary experiments described herein were performed in a modified Thermo Scientific™ Orbitrap Eclipse™ Tribrid™ mass spectrometer, wherein a gas phase proto membrane protein ion produced by a source and passed though the heated metal inlet capillary is immediately confined by a RF stacked ring ion funnel and then transmitted on though further pairs of RF multipole ion guides, which transfer the gas phase PMP ion to a quadrupole mass filter and thence, via further RF ion guides including the RF C-trap, an ion routing multipole (collision cell), another RF multipole ion guide to the high pressure region of a dual pressure region RF quadrupole linear ion trap analyser. Thus, the confinement of the gas phase proto membrane protein ion is effected by an RF ion funnel, a pair of RF multipole ion guides, a RF quadrupole mass filter, a third RF multipole ion guide, a RF C-Trap, an ion routing multipole/trapping collision cell, a fourth RF multipole ion guide and a RF quadrupole linear ion trap. The gas phase intact membrane protein ion is confined within the RF quadrupole ion trap during step (iv). To enable and effect various ion transformative processes that may optionally occur during step (v), the intact membrane protein ion and the ion or ions derived from the intact membrane protein ion may be transferred into, confined, and transferred out of the ion routing multipole/trapping collision cell (collision cell type CID), and both the high pressure region (Ion trap type resonant CID, IRMPD, ETD and proton transfer charge reduction ion-ion reactions) of the RF quadrupole linear ion trap. To effect optional sub steps of m/z isolation of the intact membrane protein ion or an ion derived therefrom in step (v) the respective ion would be confined in the high pressure region of the RF quadrupole linear ion trap.

To perform m/z analysis with the Orbitrap m/z analyser of the intact membrane protein ion or an ion derived therefrom in step (v), the respective ion is transferred to and confined in the RF C-Trap, and then radially ejected into the high orbitrap m/z analyser. To perform m/z analysis with the RF linear ion trap m/z analyser of the intact membrane protein ion or an ion derived from the intact membrane protein ion in step (v), the respective ion is transferred to and confined in the low pressure region of the RF quadrupole linear ion trap and then ejected m/z selectively and sequentially to an electron multiplier based ion detector.

This set-up is described only to illustrate the various pieces of apparatus which may be used to contain the ions produced during the method of the invention, and is not intended to be limiting. Other suitable arrangements of one or more RF ion guides, RF quadrupole mass filters, RF quadrupole ion traps, and m/z analysers can be readily envisaged by the skilled person.

The confinement of the gas phase PMP ion (and other ions produced during the method) is, of course, essential in order to spatially localise the ions to allow them to be exposed to infrared radiation of sufficient intensity and for a sufficient duration during step (iii), and to allow the gas phase intact membrane protein ions thus produced to be optionally exposed to further processes to eventually produce the derived ion(s) to be ultimately directed into an m/z analyser and detected. However, during confinement of an ion (such as the gas phase PMP ion) additional advantageous processes can be performed.

A confined ion may be exposed to a collision gas. Exposure to a collision gas can be used at different points during the method to achieve a number of different outcomes.

For instance, exposure of the gas phase proto membrane protein ion to a collision gas may remove one or more residual solvent molecules and bulk lipids molecules in a collision-induced dissociation process. So, exposure to a collision gas can assist in removing these unwanted residual solvent and bulk lipid molecules. The exposure of the gas phase PMP ion to a collision gas to remove residual solvent and bulk lipid molecules may occur before and/or during step (iv). Preferably, the exposure of the gas phase PMP ion to a collision gas to remove residual solvent and bulk lipid molecules occurs before step (iv), during step (iii).

Exposure to a collision gas can also be used to assist fragmentation of the gas phase intact membrane protein ion produced in step (iv), and/or an ion derived therefrom. Thus, the process may comprise, after step (iv), exposing the gas phase intact membrane protein ion to a collision gas in a CID process to dissociate the gas phase intact membrane protein ion and produce two or more fragments. Alternatively or additionally, the process may comprise exposing a product ion derived from the gas phase intact membrane protein ion to a collision gas in order to further dissociate the product ion and produce two or more fragments. The fragments thus produced may be, for instance, a ligand (such as a non-specifically bound ligand) dissociated from the membrane protein; a sub-unit of a membrane protein, where the membrane protein is multimeric complex; or a fragment produced by the breaking of a covalent bond within the membrane protein.

In such cases, where exposure to a collision gas is used to assist fragmentation of the gas phase intact membrane protein ion produced in step (iv), and/or a product ion derived therefrom, the ion to be fragmented must generally be confined in an RF ion trap or a multipole collision cell at relatively elevated pressure (generally above 1 mTorr). Accordingly, in such cases, the process may comprise after step (iv) transferring the gas phase intact membrane protein ion, or a product ion derived therefrom, into in an ion trap at elevated pressure or a collision cell.

The collision gas used to dissociate gas phase intact membrane protein ions or product ions derived therefrom in an RF ion trap (typically an RF quadrupole linear ion trap) via resonant trap-type CID as described above is typically a very light gas such as Hydrogen or Helium—preferably Helium—as the use of high molecular weight gases would likely interfere with the ability to use the device for m/z isolation and m/z analysis. The pressure of collision gas within an ion trap (e.g. an RF quadrupole linear ion trap) in such cases would typically be in the range of 1-10 mTorr. The collision gas used to dissociate gas phase intact membrane protein ion or product ion derived therefrom in a collision cell is generally a noble gas such as Neon, Argon, or Xenon or a relatively non-reactive molecular gas such as nitrogen (N₂) or sulfur hexafluoride (SF₆) with generally Nitrogen or Argon being preferred. The pressure of collision gas within the collision cell in such cases may be, for instance, 1-20 mTorr or greater.

Fragmentation by a collision-induced dissociation method is a less preferred method of fragmentation according to the present method. This tends to produce a lower yield of sequence fragment ions and a relatively higher yield of side chain and loss fragment ions when applied to intact membrane protein ions. Nonetheless, fragmentation by CID can be a useful technique and is an example of an ion transformative process as described herein. Other, more preferred ion transformative processes (particularly IRMPD) are described below; these methods are also performed while the ion is confined, for instance in an RF quadrupole linear ion trap or in a collision cell.

Particularly importantly, though, an effect of the presence of a collision gas in the method of the invention is to provide a cooling effect during step (iv). During step (iv), if the gas phase PMP ion is stored in the presence of a collision gas, near thermal energy collisions with the gas atoms or molecules extracts vibrational energy from the gas phase PMP ion and counteracts the heating effect of the infrared irradiation. It is believed that this enables the gas phase PMP ion to reach some sort of equilibrium level of vibration excitation upon exposure to infrared irradiation. Thus, controlling the gas pressure and the nature of the gas present during step (iv) allows further control over heating of the gas phase proto membrane protein ions, and can be used to optimise the production of the gas phase intact membrane protein ion. Accordingly, step (iv) may be preferably performed while the gas phase proto membrane protein ion is confined in the presence of a collision gas (for instance, in an RF quadrupole linear ion trap).

The suitable collision gases which may be used to cool the gas phase proto membrane protein ion as described above would include Nitrogen, Hydrogen, or a noble gas such as Helium, Argon, or Neon; preferably Hydrogen or more preferably Helium if the gas phase PMP ion is confined within a RF quadrupole linear ion trap analyser. The pressure of collision gas in such cases may be, for instance, less than 10 mTorr; for example from 5 to 10 mTorr, typically around 7 mTorr.

For any particular ion confinement device utilized to confine the gas phase proto membrane protein ion, the properties of the irradiating infrared photon beam (wavelength, beam power, beam diameter etc.) in step (iv) of a method according to the invention, the optimal setting should be readily determined by experiment.

The advantages of confining ions produced during the method do not only stem from the ability to expose such confined ions to a collision gas. In addition, the confinement of ions often allows specific ion m/z ranges to be selected such that ions having m/z outside of the selected ranges are not confined and hence are eliminated. All RF ion confinement devices have m/z dependence in the confinement of ions. RF quadrupole field devices such as RF quadrupole m/z filters and RF quadrupole linear ion traps can be operated to have very sharp transitions between ranges of m/z confinement and m/z ejection or non-confinement. The numerous techniques for effecting selection of specific m/z ranges with RF quadrupole field devices are well known in the art. Depending upon the specific device, the range for m/z selection may be narrower than 0.1% of the m/z at the midpoint of the selected m/z range. Selection of a range of mass-to-charge ratios, if performed on an ion beam is often referred to in the art as “mass selection” and, with somewhat more rigor, as “m/z selection”. Selection of a specific mass-to-charge ratio, or range of mass-to-charge ratios and elimination of ions having m/z values outside of that selected range when performed on a trapped population of ions is often referred to in the art as “mass isolation” and, with somewhat more rigor, as “m/z isolation”. For the purposes of this specification m/z isolation and m/z selection can be considered equivalent terms.

m/z isolation may be performed in connection with a gas phase proto membrane protein ion; a gas phase intact membrane protein ion; or an ion derived from a gas phase intact membrane protein ion. This in turn allows analysis of a gas phase proto membrane protein ion or gas phase intact membrane protein ion having a specific m/z ratio, or falling within a specific m/z range. This ability to m/z select an ion or group of ions to be subjected to further transformational processes allows more detailed analysis of the structure and composition of the membrane protein therein. After the production of any ion, the process may comprise a step of performing m/z isolation in order to isolate the ion or ions having a particular m/z or range of m/z values. This may be referred to as m/z isolation, or m/z selection.

m/z selection of a gas phase proto membrane protein ion, a gas phase intact membrane protein ion or an ion derived from a gas phase intact membrane protein ion can be performed, for instance, rather crudely in an RF ion guide and with much more precision in an RF quadrupole linear ion trap analyser or a quadrupole m/z filter. The ions having unwanted m/z ratios may be ejected from the RF ion guide, RF quadrupole linear ion trap or RF quadrupole m/z filter. This may comprise actively adjusting the magnitudes and frequency composition, or, in some instances, applying additional the types of voltages to the RF ion guide, RF quadrupole linear ion trap or RF quadrupole m/z filter electrodes in order to expel particular m/z ranges of ions, or may simply comprise allowing the ions having unwanted m/z ratios to escape.

In practice, the process involves pre-determining the desired range of m/z ratios to be retained, and then ejecting the ion(s) having an m/z ratio outside that range from an RF ion guide, RF quadrupole linear ion trap, RF quadrupole m/z filter or other device in which the ion is confined.

For instance, in some embodiments of the method described herein, during step (iii) and/or step (iv) the gas phase PMP ion and/or the gas phase intact membrane protein ion is held in an ion trap or transmitted through an ion guide or mass filter, and the method comprises:
isolating the gas phase PMP ion and/or the gas phase intact membrane protein ion by ejecting ions outside one or more pre-determined m/z windows from the RF ion guide, RF quadrupole linear ion trap, RF quadrupole m/z filter.

This m/z selection step can of course be repeated prior to and/or after the ion transformative process(es) performed on the gas phase intact membrane protein ion and the ion(s) derived therefrom. For instance, in some embodiments of the method described herein, after step (iv) the gas phase intact membrane protein ion or an ion derived therefrom is held in an ion trap or transmitted through an ion guide or a mass filter, and the method comprises: m/z isolating or m/z selecting the gas phase intact membrane protein ion or the ions derived therefrom by ejecting ions of m/z outside one or more pre-determined m/z ranges from the ion trap, ion guide or mass filter (generally wherein the ion trap, ion guide or mass filter is an RF ion guide, RF quadrupole linear ion trap, or RF quadrupole m/z filter).

Step (iv)—Irradiation

Step (iv) of the method involves detaching the one or more residual solvent molecules and one or more non-specifically bound bulk lipid molecules from the intact membrane protein by irradiating the confined gas phase proto membrane protein ion with infrared radiation from a laser source to produce a gas phase intact membrane protein ion. The gas phase intact membrane protein ion is as described above.

The inventors have found that irradiation using infrared radiation from a laser source can be used to selectively fully “evaporate” the solvent molecules and, surprisingly, bulk lipid molecules from the gas phase proto membrane protein ion to produce the gas phase intact membrane protein ion. The inventors have found that the use of such IR activation generally produces a higher yield of membrane intact membrane protein ions from a plurality of gas phase PMP ions than by use of well optimised conventional inlet CID.

Without wishing to be bound by theory, it is speculated that the infrared radiation is approximately resonant or near-resonant with one or more vibrational modes of the bulk lipid molecules. Accordingly, the infrared radiation may be more efficiently absorbed by those molecules, leading to dissociation of those molecules from the gas phase proto membrane ion, in preference to absorption of radiation by the membrane protein itself and its consequent dissociation. It is speculated that this resonant or near-resonant overlap of the photon wavelength and the absorption bands of covalent bonds in the bulk lipids may enable higher absorption of the IR radiation per vibrational degree of freedom for the bulk lipids than for the membrane protein. Thus with an appropriate choice of laser power setting and exposure time, this differential heating causes the relatively lower affinity bulk lipid molecules to detach from the membrane protein without dissociation of the membrane protein itself during step (iv).

It is further speculated that the solvent molecules have lower binding affinity than the bulk lipids and even if the solvent molecules have about the same absorbance of IR energy per vibrational degree of freedom as the membrane proteins, an IR radiation flux sufficient to detach the bulk lipids is more than sufficient to detach the solvent molecules.

Accordingly, the infrared irradiation selectively detaches bulk lipid molecules and residual solvent molecules, leading to a high yield of gas phase intact membrane protein ions. As explained above, the gas phase intact membrane protein ion is free of bulk lipid molecules and is free of residual solvent molecules.

Both fixed-wavelength and variable-wavelength infrared laser sources are known. Fixed-wavelength laser sources generally have the advantage of high power output, ease of use, high commercial availability and much lower cost. Variable-wavelength laser IR sources would allow for optimisation of the IR wavelength used in the method at hand. It is anticipated that it would be particularly advantageous, for example, to be able to select the output wavelength of infrared radiation from the IR laser source which has the best resonant overlap with the bulk lipid molecules present in the gas phase proto membrane protein ions generated in the particular analysis. That may optimise the selective loss of bulk lipid molecules from the gas phase PMP ions and enable maximisation of the probability of producing a gas phase intact membrane protein ion from each gas phase proto membrane protein ion.

Thus, in a preferred embodiment of the method, a wavelength of the infrared radiation output from the laser source is chosen to preferentially excite one or more vibrational modes of one or more bonds within the non-specifically bound bulk lipid molecules. An appropriate wavelength may be selected by varying the wavelength of the infrared radiation and repeating the disclosed method until the intact membrane protein ion can be detected after step (iv).

The laser source may be a continuous wave laser source or a pulsed laser source. Pulsed laser sources generally have the property that the output pulse length, pulse frequency, and, potentially, pulse output power may be adjusted and thus enabling, in the context of the performing the disclosed method, optimization of the yield of intact membrane protein ions from the irradiated gas phase proto membrane protein ions though repetitions of method and varying laser parameters. A pulsed laser source may be advantageous in that it may be able to deposit larger amounts of vibrational energy into the gas phase proto membrane protein ion and, specifically, the preferentially absorbing bonds of the attached bulk lipid molecules in very short intervals of time. This may promote rapid heating of the bulk lipids and their detachment while limiting transfer of vibrational energy from the non-covalently associated bulk lipids to the membrane protein and thereby reduce dissociation of the membrane during removal of the bulk lipids.

The experimental studies exemplified herein utilized a continuous fixed wavelength infrared laser. This laser was an CO₂ gas laser designed for industrial marking and engraving applications with a nominally 10.6 μm output wavelength and with a specified nominal maximum output power of 60 watts (uncalibrated). The laser output power could be controlled with an analogue voltage which enabled the power output to be adjusted from 0% to 100% of maximum output. A pair of adjustable mirrors were used to align laser beam with the central axis of the three segment high pressure region of the RF quadrupole linear ion trap. During these initial pioneering experiments, the RF quadrupole field generating potentials and axially confining segment DC bias potentials were applied to the electrodes of the high pressure region of RF quadrupole linear ion trap during the irradiation step (iv) of the method disclosed, such that the cloud of gas phase PMP ions should have been radially confined to within a 1 mm or less radius of the device's axis and confined in a region along that axis approximately corresponding to the ˜37 mm long central segment of the device. The IR laser beam was focused to locate the beam waist within the high pressure quadrupole linear ion trap and the beam diameter, according to the relatively crude methods that were available to measure it, was fairly uniform with a 2-3 mm diameter radius along the portion of the axis of the device where that gas phase PMP ions were confined. The laser output power control signal was under full control of the instrument's embedded control system and laser output could be turned on to the desired output power to initiate irradiation of the gas phase PMP ions and back to zero out at the end of the irradiation period. According to the laser specifications the response time of the laser output to transition in either direction between effectively no output power and a stable output power was well less than 1 msec. Fixed wavelength IR lasers with similar specifications are readily commercially available. Common nominal output wavelengths for continuous wave CO₂ gas lasers in the same output power ranges are 9.3 μm, 10.2 μm, and 10.6 μm.

Thus, in some cases, the infrared radiation from the laser source may be pulsed radiation and in other cases the infrared radiation from the laser source may be continuous-wave radiation. Preferably, the infrared radiation from the laser is continuous wave radiation.

The wavelength of the radiation from the laser source is in the infrared region of the electromagnetic spectrum. Thus, the wavelength of the infrared radiation is generally from 780 nm to 1 mm. Given that the invention relies on the absorption of infrared radiation by vibrational modes of a lipid molecule, the wavelength of the infrared radiation is preferably a wavelength overlapping with one or more vibrational modes of the bulk lipid molecule. The vibrational modes of the bulk lipid molecules tend to be in the micron range. Thus, preferably, the wavelength of the infrared radiation is in the range of 1.4 μm to 1 mm. More preferably the wavelength is from 3 μm to 100 μm, e.g. from about 5 μm to about 50 μm, such as from about 8 to about 15 μm and most preferably the wavelength is between 9 and 11 μm.

Conveniently, the laser source may be a CO₂ laser. Thus, the wavelength of the infrared radiation from the laser source may have a principal wavelength of one or more of the following wavelengths: 10.6 μm, 10.2 μm, 9.6 μm, and 9.3 μm. Particularly preferably, the wavelength of the infrared radiation from the laser source has a principal wavelength of 10.6 μm.

The irradiation step of the present method is intended to dissociate all bulk lipid molecules (in either neutral or ionic form) from the gas phase proto membrane protein ion and, if necessary, to any remove residual solvent molecules. Further it is also intended that the irradiation step does not dissociate the membrane protein. Rather, step (iv) produces a gas phase intact membrane protein ion. The gas phase intact membrane protein ion produced may retain any and preferentially all complexed specifically bound ligands such as a drug, RNA strand, protein specific lipids, post translational modifications and so on.

Accordingly, the degree of vibrational activation of the gas phase PMP ion—as controlled by factors including irradiation power-during step (iv) is generally distinctly lower than the level of vibrational activation required to effect dissociation of covalent bonds within the membrane protein via conventional IRMPD processes.

It should be understood that for a given level of vibrational activation of the gas phase PMP ion for a specific time interval and collision gas and collision gas pressure, both dissociation of the non-covalent bonds and covalent bonds have some probability of occurring. As discussed above, the binding energies of the non-covalent bonds that hold the bulk lipids and the solvent molecules within the gas phase PMP ion are generally expected to be lower than binding energies of the non-covalent bonds which bind specifically bound ligand molecules to the membrane protein. They are almost certainly lower than binding energies of the covalent bonds within the membrane protein. Consequently, at suitably low levels of vibrational activation, dissociation of non-covalent bonds which bind solvent molecules and the bulk lipids to the membrane protein are much more probable within the activation time interval than cleavage of any one non-covalent or covalent bond of the membrane protein.

To the extent that preferential absorption of IR radiation by the bulk lipids increases the relative vibrational activation per vibrational degree of freedom (temperature) of the bulk lipids in a gas phase proto membrane protein ion during the irradiation period, this will further bias the probability of the various dissociation pathways toward effecting detachment of the bulk lipids without dissociation of the membrane protein.

Hence the probability of producing a gas phase intact membrane protein ion from a confined gas phase proto membrane protein ion can be optimised by using relatively lower laser powers in comparison to those favoured to effect dissociation of the membrane protein's covalent and non-covalent bonds in combination.

Where a plurality of gas phase proto membrane protein ions are confined in step (iii), that plurality of ions is typically irradiated during step (iv) to produce a plurality of gas phase intact membrane protein ions. This may also be referred to as a population of gas phase intact membrane protein ions. Each gas phase intact membrane protein ion within the plurality of gas phase intact membrane protein ions is as described herein.

Each gas phase intact membrane protein ion within a plurality of gas phase intact membrane protein ions may be the same or different. In some cases, each gas phase intact membrane protein ion has an identical chemical structure. Alternatively, each gas phase intact membrane protein ion within the plurality of gas phase intact membrane protein ions may have a different chemical structure. For instance, the plurality of gas phase intact membrane protein ions may comprise two or more gas phase intact membrane protein ions, wherein each gas phase intact membrane protein ion has a differing chemical structure.

The intact membrane protein ions present may also differ in their charge state. In some cases, each gas phase intact membrane protein ion within the plurality of gas phase intact membrane protein ions may have an identical charge. Alternatively, each gas phase intact membrane protein ion within the plurality of gas phase intact membrane protein ions may have a differing charge state (even where each intact membrane protein has the same amino acid sequence, or is chemically identical). For instance, the plurality of gas phase intact membrane protein ions may comprise two or more gas phase intact membrane protein ions, wherein each gas phase intact membrane protein ion has a differing charge state.

During step (iv), where a population of confined gas phase proto membrane protein ions is irradiated, ideally all of the confined gas phase PMP ions should be exposed to the same intensity of light during the irradiation step (iv). If so, the probability of producing a gas phase intact membrane protein ion is the same for all of the confined gas phase proto membrane protein ions are chemically and structurally identical. It is preferred that the optical system that delivers the IR light is configured to provide a high degree of uniformity in photon flux within the volume where the gas phase PMP ions/gas phase IMP ions are confined. Further, to minimise the maximum output power and therefore the size and cost of the IR laser it is preferred to maximize photon flux throughout the region where the gas phase PMP ions are localized during the irradiation period of step (iv) for any given laser output setting. Optimal parameters may be determined by routine trial and error. For instance, parameters may be determined by performing repeated experiments and adjusting the laser output power, the irradiation period and the cooling collision gas pressure (within the capabilities of the apparatus used) to optimise the yield of gas phase intact membrane protein ions.

The gas phase proto membrane protein ion (or, where present, the plurality of gas phase proto membrane protein ions) are confined in step (iii). They are typically localised within a volume defined in space. Thus, step (iii) may comprise confining the gas phase PMP ion (or the plurality of gas phase PMP ions) within a volume. As explained above, during step (iv) it is preferred that the infrared radiation from the laser source has a uniform photon flux within said volume. Thus, the laser source may be configured to provide a uniform photon flux within said volume.

For instance, the present method typically comprises performing the irradiation for a period of up to 1 second, preferably for a period of less than 50 ms. The method may comprise performing the irradiation for a period ranging from 1 us to 50 ms. The period during which irradiation with the infrared radiation from a laser source is performed may be referred to as the “period of irradiation”. For instance, a particular apparatus operating according to the method may be configured to perform irradiation for a period of a duration that may range from 100 us to 20 ms.

It should be understood that the gas phase intact membrane protein ion may form during the period of irradiation, or shortly afterwards (following the absorption of energy while the gas phase proto membrane protein ion remains sufficiently vibrationally activated). That is, the one or more residual solvent molecules and the one or more non-specifically bound bulk lipid molecules may detach from the intact membrane protein in the gas phase proto membrane protein ion during the period of irradiation. Alternatively, they may detach from the intact membrane protein after irradiation. Accordingly, whilst the irradiation is applied to a confined gas phase proto membrane protein ion, the composition of the gas phase proto membrane protein ion may change during the period of irradiation. For instance, the gas phase proto membrane protein ion may be altered by the detachment of one or more residual solvent molecules and one or more non-specifically bound bulk lipid molecules. The gas phase intact membrane protein ion may be formed from the gas phase proto membrane protein ion during the period of irradiation.

Where the irradiation is performed on a population of gas phase proto membrane protein ions, the rate at which residual solvent molecule(s) and non-specifically bound bulk lipid molecule(s) detach from the intact membrane protein may differ between gas phase proto membrane protein ions. By way of example, consider a population comprising two gas phase proto membrane protein ions where each of said gas phase proto membrane protein ions comprises the identical intact membrane protein, the same numbers and types of residual solvent molecules and the same number and types of non-specifically bound bulk lipid molecules. Even though the two said gas phase proto membrane protein ions are subjected to the same irradiation conditions during step (iv), the detachment of said residual solvent molecule and said non-specifically bound bulk lipid molecules will likely occur at different times.

Accordingly, where the irradiation is performed on a population of gas phase proto membrane protein ions, the irradiation parameters (such as the irradiation period and the laser output power) are typically selected in order to maximise the number of gas phase intact membrane ions which is produced from the population of gas phase proto membrane protein ions. In other words, said parameters are typically selected in order to maximise the size of the final population of gas phase intact membrane protein ions.

For instance, the present method may comprise operating the laser at 12% or less of its available output power, such as 10% or less of its available output power during the irradiation step (iv). By contrast, in order to effect efficient IRMPD of ions identically confined under identical conditions with an irradiation event of the same duration would typically require operating the laser at more than 10% of available laser output power, such as 15% or more of the available laser output power.

The specific irradiation parameters to be used during the irradiation of step (iv) are generally best determined by trial and error. The key irradiation parameters include the length of time during which the irradiation is performed (the “period of irradiation”); the power of the laser source (for example 3-12% of the available power, e.g., corresponding to approximately 1.8-7.2 W of a 60 W continuous wave infrared CO₂ laser), and the wavelength of the laser source.

In some embodiments the laser power is about 0.1 to about 10% of the power of a 60 W continuous wave infrared CO₂ laser (e.g. from about 0.06 W to about 6 W), such as from about 1% to about 8% (e.g. from about 0.6 W to about 4.8 W), e.g. from about 2% to about 6% (1.2 W to about 3.6 W). For example, such power levels are in some embodiments useful to liberate intact membrane proteins from vesicles derived from their native lipid layer. In some embodiments the laser power is from about 10% to about 30% of the power of a 60 W continuous wave infrared CO₂ laser (e.g. from about 6 W to about 18 W), such as from about 12% to about 20% (e.g. from about 7.2 W to about 12 W), e.g. from about 15% to about 18% (e.g. about 9 W to about 10.8 W). For example, such power levels are in some embodiments useful to liberate fragment the polypeptide chain of an intact membrane protein or fragment thereof.

Thus, in some embodiments the irradiation of step (iv) may comprise, for example, irradiating at a wavelength of from about 3 μm to 100 μm, e.g. from about 5 μm to about 50 μm, such as from about 8 to about 15 μm, e.g. between about 9 and about 11 μm, at a power corresponding to about 0.1% to about 30% of a 60 W continuous wave infrared CO₂ laser (e.g. at about 0.06 to about 18 W), e.g. from about 1% to about 8% (e.g. from about 0.6 W to about 4.8 W), e.g. from about 2% to about 6% (1.2 W to about 3.6 W); or from about 12% to about 20% (e.g. from about 7.2 W to about 12 W), e.g. from about 15% to about 18% (e.g. about 9 W to about 10.8 W); for a period of up to about 1 second, preferably for a period of from about 1 us to about 50 ms, such as from about 100 us to 25 ms, e.g. from about 5 ms to about 25 ms.

Additional irradiation parameters which may be varied include the size and position of the beam waist and the divergence of the IR photon beam relative to the volume wherein the gas phase proto membrane protein ion (or the population of gas phase proto membrane protein ions) are localized when confined during the irradiation step (iv). If a pulsed laser is used, the laser's output pulse power, the pulse length, and repetition rate are all parameters that may potentially be adjusted to promote the formation of a gas phase intact membrane protein ion.

Where a population of gas phase proto membrane protein ions is confined during step (iii), said parameters may be optimised to maximise the yield of a population of gas phase intact membrane protein ions from the population of gas phase proto membrane protein ions confined in step (iii).

In some embodiments, the method comprises determining irradiation parameters to be used in step (iv) by repeatedly performing the process and varying the power of the laser radiation and/or the time period during which the irradiation is performed until the gas phase intact membrane protein ion can be detected after step (iv). When far from optimal irradiation parameters are used, it is generally not possible to observe a m/z peak corresponding to a population of a particular gas phase intact membrane protein ion above the baseline in the m/z spectrum (which contains signal from the broad distribution in m/z of residual gas phase proto membrane protein ions, various gas phase matrix ions that do not comprise membrane protein, and electronic noise). Suitable irradiation parameters are obtained once a m/z peak corresponding to a gas phase IMP ion can be distinguished from the spectral baseline.

Determination of these irradiation parameters is a matter of routine optimisation which can readily be performed by the skilled person. Such optimisation would typically involve multiple trial experiments using various combinations of different irradiation parameters (including irradiation time and laser output power settings) to determine the irradiation parameter settings which optimise the production of the gas phase intact membrane protein ion. In particular, the optimisation may be performed to determine the combination of irradiation parameters which maximises the population of gas phase intact membrane protein ions produced in step (iv).

One or more figures of merit may be used in evaluation of the m/z spectra produced in these trial experiments. One such figure of merit may be the abundance of m/z peaks in said m/z spectra which correspond to detected intact membrane protein ions. Another such figure of merit may be the number of clearly observable m/z peaks in said m/z spectra that correspond to different intact membrane protein ions of interest. The optimization experiments can be performed manually by choosing the irradiation parameter settings for each trial experiment.

There are many methods known in the art for full or partial automation of optimization of this sort. Parameters of a modern, highly computerised, mass spectrometer's function can be optimised based on detected ion signal and the attributes of the m/z spectra produced by the instrument using automated procedures that “tune”, or “calibrate” the instrument. Similarly, the optimization experiments and determination of the optimal parameter settings for step (iv) maybe partially or fully automated.

Step (v)—m/z Analysis and Detection

Step (v) of the method comprises m/z analysing and detecting the gas phase intact membrane protein ion and/or an ion derived therefrom.

The method described herein is a mass spectrometric method. Thus, step (v) comprises determining the m/z of said gas phase intact membrane protein ion and/or an ion derived therefrom.

In practice, the method typically comprises measuring signal intensity as a function of mass-to-charge (m/z) ratio, by any suitable measurement means. Accordingly, step (v) generally involves transferring the gas phase intact membrane protein ion and/or an ion derived therefrom to a m/z analyser and detector, and m/z analysing and detecting the said gas phase intact membrane protein ion and/or an ion derived therefrom. Any suitable combination of m/z analyser and detector may be used. By way of illustration, a high field orbitrap analyser was employed in exemplary methods described herein. In other instances, an RF quadrupole linear ion trap analyser and its associated conversion dynode-electron multiplier based ion detector in the same instrument could be used. Other examples of m/z analyser-detector combinations that would be suitable for use with the method described herein would a Time-of-flight analyser and associated ion detector as well as a Fourier Transform Ion Cyclotron Resonance (FT-ICR) analyser.

Performing step (v) will typically produce an m/z spectrum, or m/z spectral data. The m/z spectrum may be analysed to determine the m/z of any ion produced by the process described herein.

To assist with analysis and characterisation, after the production of any ion, the process may comprise a step of performing m/z isolation in order to isolate the ion or ions having a particular m/z or range of m/z values. The isolated ion or ions can be subjected to an ion transformative process, or may be m/z analysed and detected.

### Ion Transformative Processes

It is possible to m/z analyse and detect the gas phase intact membrane protein ion produced in step (iv), and thus to determine its m/z ratio. However, where the identity of a particular observed gas phase intact membrane protein ion is unknown, it is advantageous to subject the gas phase intact membrane ion and/or one or more product ion(s) derived therefrom to one or more ion transformative processes (such as fragmentation or charge reduction). It is then possible to m/z analyse and detect the various product ions thus derived from the gas phase intact membrane protein ion. Such processes, when performed on a population of chemically identical gas phase intact membrane protein ions with identical structure and molecular composition, can yield information about the structure and chemical identity of the gas phase intact membrane protein ions.

Thus, in a preferred embodiment of step (v), the gas phase intact membrane protein ion is subjected to one or more ion transformative processes to produce at least one product ion, and the product ion is m/z analysed and detected. A “product ion” refers to an ion produced from another ion. A product ion is produced by an ion transformative process, performed on another ion. In this context a first-generation product ion is an ion which is produced directly from a gas phase intact membrane protein ion.

In other preferred embodiments of step (v) the product ion may be subjected to one or more ion transformative processes to produce one or more next-generation product ion(s). Any product ion (that is, any product ion derived from a previous generation product ion), may be m/z analysed and detected. For instance, the gas phase membrane protein ion may be fragmented to produce a product ion, and this first-generation product ion may be subject to further fragmentation though one or more ion transformative processes. Any next-generation product ion formed through this succession of transformative processes may be m/z analysed and detected.

Thus, step (v) may comprise m/z analysing and detecting the gas phase intact membrane protein ion, and/or an ion derived therefrom, in a tandem mass spectrometry method.

Accordingly, by way of illustration, step (v) may comprise fragmenting the gas phase intact membrane protein ion to produce a product ion which is a fragment ion. This fragment ion may then be directly m/z analysed and detected. Alternatively, this fragment ion may be charge-modified prior to m/z analysis and detection. For instance, the charge of a fragment ion may be reduced in order to increase its m/z ratio, as this can increase the separation in m/z between product ions that may be generated when a population of chemically identical gas phase intact membrane protein ions are simultaneously transformed according to step (v) of the method.

In yet another alternative, a fragment ion may itself be fragmented, to produce a further fragment ion, of the next generation. The further fragment ion can be m/z analysed and detected or it can be subjected to one or more further ion transformative processes. The further fragment ion may be charge-modified and then m/z analysed and detected (e.g. its charge may be reduced prior to m/z analysis and detection), or the further fragment ion may be further fragmented. That is, more than two fragmentation processes may be performed during step (v). For example, 3, 4, 5, 6, 7, 8, 9 or 10 fragmentation processes may be performed during step (v) to yield a plurality of fragment ions.

Multiple fragmentation processes will lead to large numbers of fragment ions. Accordingly, where fragmentation processes are performed, m/z selection of a desired ion or several ions is generally performed between fragmentation processes and also optionally prior to m/z analysis and detection. The m/z selection process is discussed above in “Step (iii)—confinement”.

However, the ion transformative processes need all not necessarily be fragmentation processes. Step (v) may comprise subjecting the gas phase intact membrane protein ion to one or more ion transformative processes to produce a product ion (specifically, a first-generation product ion). This first-generation product ion may then be directly m/z analysed and detected. Alternatively, the first-generation product ion may be subjected to one or more further ion transformative processes, to produce a second generation product ion. The second-generation product ion can be detected or be subjected to further ion transformative processes. That is, more than two ion transformative processes may be performed during step (v). For example, 3, 4, 5, 6, 7, 8, 9 or 10 ion transformative processes may be performed during step (v).

Thus, step (v) may comprise
(va) subjecting the gas phase intact membrane protein ion to a transformative ion process to produce a first-generation product ion; and
(vb) m/z selecting and optionally m/z analysing and detecting the first-generation product ion.

By “m/z analysing” is meant that the ion is subjected to an m/z-dependent dispersion process. The process allows the mass-to-charge ratio of the detected ion to be determined. The process generally disperses the ion in space, or in time, or in frequency of motion, dependent on the ion's m/z. This means that when the ion is detected, its m/z may be determined with reference to its location of detection, its time of arrival at the detector, path stability as determined by the applied voltages to the m/z analyser electrodes or the frequency of its motion indicated by the frequency of the a detected signal it induces. The m/z dependent dispersion process typically involves an interaction with an electromagnetic field. One example involves the interaction of an ion with an electric field within a time of flight mass spectrometer. In that case, the ion is given kinetic energy by an electric field, and is thus accelerated to a velocity dependent on its m/z. Consequently, the time of flight required to arrive at the detector after interaction with the electric field depends on the ion's m/z and allows its m/z to be determined.

In some cases, more than one first-generation product ion is produced. In such cases, step (vb) comprises m/z selecting at least one of the first-generation product ion. For instance, step (vb) may comprise m/z selecting all first-generation product ions produced in step (vb), and detecting and m/z analysing those first-generation product ions. Alternatively, one or more of the first-generation product ions may be subjected to one or more further ion transformative processes prior to m/z analysis and detection.

Thus, step (v) may further comprise, after step (vb),
(vc) subjecting the first generation product ion to an ion transformative process to produce a second generation product ion; and
(vd) m/z selecting the second generation product ion, and optionally m/z analysing and detecting the second generation product ion.

In some cases, more than one second-generation product ion is produced. In such cases, step (vd) comprises m/z selecting (and optionally detecting and m/z analysing) at least one of the second generation product ions. For instance, step (vd) may comprise detecting and m/z analysing all second generation product ions produced in step (vc). Alternatively, one or more of the second generation product ions may be m/z selected, and subsequently subjected to one or more further ion transformative processes prior to m/z analysis and detection.

It will be appreciated that further ion transformative processes and optional detection processes may be performed to produce further product ions, and these processes could be designated step (ve), (vf), and so on.

Where the ion transformative process is a fragmentation process, the ion(s) produced by fragmentation processes during step (v) may be, for instance, a ligand (such as a non-specifically bound ligand) dissociated from the membrane protein; a sub-unit of a membrane protein, where the membrane protein is multimeric complex; or a fragment produced by the breaking of a covalent bond within the membrane protein. The fragmentation process or processes performed during step (v) may additionally produce uncharged fragments.

In reality, the process is typically performed on a plurality of gas phase IMP ions, and a plurality of first-generation product ions is typically produced. Similarly, subsequent ion transformation processes typically produce a plurality of next-generation product ions. Each product ion in the plurality of next-generation product ions may be the same or different.

A wide variety of ion transformative processes (particularly fragmentation processes) are envisaged for any fragmentation process or processes performed during step (v). For instance, the dissociative ion transformation may be achieved by one or more of the following methods: infrared multiphoton dissociation (IRMPD), electron transfer dissociation, activated ion electron transfer dissociation, electron capture dissociation, UV photodissociation and collision-induced dissociation. In general, among these, IRMPD is preferred as the inventors have found that it generally provides a high yield of sequence informative ions from membrane gas phase membrane protein ions of regardless of their molecular weight.

Thus, in some embodiments, step (v) may comprise:
(va) fragmenting the gas phase intact membrane protein ion by IRMPD to produce an ion which is a fragment ion; and
(vb) m/z selecting the fragment ion.

In particular embodiments, step (v) may comprise:
(va) fragmenting the gas phase intact membrane protein ion by IRMPD to produce an ion which is a fragment ion; and
(vb) m/z selecting and optionally m/z analysing and detecting the fragment ion by a CDMS method.

The selection of the most appropriate ion transformative process during step (v) may depend on the type of product (e.g. a fragmentation product) desired. For instance, some fragmentation techniques are better-suited to breaking non-covalent bonds (as may be desired if the gas phase intact membrane protein ion itself is to be fragmented by removing any ligands and/or dissociating any associated sub-units within a multimeric membrane protein). Such techniques include, IRMPD, collision-induced dissociation (including RF Ion Trap type resonant collision induced dissociation, and beam type/collision cell type low energy collision induced dissociation). Surface induced dissociation is also an alternative, although less preferred.

By contrast, where it is intended to dissociate covalent bonds, suitable techniques include IRMPD, collision-induced dissociation (including trap-type CID, and low-energy beam-type CID), ultra-violet photodissociation (UVPD) and electron transfer dissociation. It may be desirable to utilize some form of fragment ion protection (e.g. product ion parking, see US 2010/0084548 and Ugrin et al., Journal of the American Society of Mass Spectrometry, 2019, vol. 30 pp 2163-2173, which are incorporated herein by reference) to limit the extent of generation of internal (non-C- and N-terminal including products) where IRMPD, UVPD or ETD are employed.

Thus, in some embodiments, step (v) may comprise:
(va) fragmenting the gas phase intact membrane protein ion by collision-induced dissociation to produce an ion which is a fragment ion;
(vb) m/z selecting the fragment ion;
(vc) fragmenting the fragment ion by IRMPD to produce a further fragment ion; and
(vd) m/z analysing and detecting said further fragment ion.

As mentioned above, the method may comprise, prior to any step wherein an ion is detected, or fragmented, modifying the charge of an ion (which may be referred to as a precursor ion) to produce the said ion to be detected or fragmented.

For example, step (v) may comprise:
(va) fragmenting the gas phase intact membrane protein ion (preferably by IRMPD) to produce a fragment ion;
(vb) m/z selecting a range of m/z that includes the m/z of said fragment ion thereby m/z selecting said fragment ion;
(vc) reducing the charge of said fragment ion to produce a charge reduced fragment ion;
(vd) detecting and m/z analysing the charge reduced fragment ion.

Charge reduction is generally effected by proton transfer ion-ion reactions, but it is possible (although less preferred) to achieve this by other means such as (but not limited to) electron transfer ion-ion reactions or electron capture (ion-electron) reactions.

### Further Analysis

So far the method has been described and defined in terms of the genealogy of a single m/z analysed and detected ion derived from a single proto membrane ion produced in step (ii) of the method. In practice the method is performed to generate and analyse populations of ions simultaneously. Depending on the m/z analyser used and various other instrumental considerations, the number of ions m/z collectively analysed and detected in a single iteration of the method may range from single digits to more than 100,000. Further, the method is generally repeated multiple times generating and analysing multiple populations of ions from the same sample. In general, the aggregated data from all of these applications of the method will, through the m/z analysis and detection performed in step (v) of the method, when digitized, be a set of m/z ion signal abundance pairs data and may be plotted as a m/z spectrum. When presented as a m/z spectrum, this data will exhibit m/z peak(s) indicative of the detection of the gas phase intact membrane protein ions of various types and/or product ions derived therefrom. A key purpose of the invention is that those m/z spectra can be used to determine structural and chemical information concerning one or more membrane proteins in a provided sample.

For example, at a simple level, analysis of the m/z spectra obtained may allow chemical identification of any ligands bound to the membrane protein in the gas phase intact membrane protein ion. It may also provide information about the relative abundance of different proteoforms of the same protein in the provided sample or the relative abundance of different proteins in the provided sample. Thus, in general terms, the method may further comprise:

(vi) utilizing the m/z spectral data obtained in step (v) (for instance, analysing the m/z spectrum resulting from the m/z analysis and detection of the fragment ions of a particular membrane protein ion type produced by multiple applications of the method on a provided sample) to provide information about the structure and/or identity, and or abundance of an intact membrane protein in the provided sample.

In particular, step (vi) may involve utilizing the m/z-ion signal abundance data obtained in step (v) in order to identify the membrane protein, or in order to obtain information on the amino acid sequence of the membrane protein, or in order to identify a binding partner of the membrane protein. The membrane protein here is the membrane protein present during the process of the invention; as noted above, the process may be repeated to identify multiple different membrane proteins present in a sample. Automated software tools for assisting the analyst in extracting such information from the aggregated m/z-ion abundance signal data (m/z spectral data) are known in the art. These data analysis tools are highly sophisticated and can allow the extraction of a great detail of information concerning an intact membrane protein (multiple copies) in the sample from which the m/z analysed and detected ions which yielded the m/z spectrum were derived. Such information, depending upon the specific experiment (the specifics of the ion transformations performed in step (v) of the method that produced the m/z-detected ion abundance signals that were aggregated to produce the m/z spectral data), may include the amino acid sequence of all or part of the intact membrane protein, and/or the location and/or type of a post-translational modification of the intact membrane protein.

Suitable software for extracting such information such m/z spectral data includes Protein prospector (UCSF), Byos/Byonic (Protein Metrics), Proteome Discoverer (Thermo Scientific), TD Validator, Prosight Native, Prosite Lite, and all other suites from Proteinaceous Inc, and MASH Explorer (University of Wisconsin-Madison).

### EXEMPLARY EMBODIMENTS

The particular features of each of steps (i) to (v) described above may be combined to produce particularly preferred embodiments of the method. Examples of such combinations are as follows.

A method of analysing a sample comprising multiple copies of an intact membrane protein, the method comprising:
(i) providing a sample comprising a non-synthetic biological membrane material comprising multiple copies of the intact membrane protein, a solvent, and bulk lipid molecules;
(ii) ionising the sample to produce a plurality of gas phase proto membrane protein ions, each of said gas phase proto membrane protein ions comprising the intact membrane protein non-specifically bound to one or more bulk lipid molecules and to one or more residual solvent molecules;
(iii) confining the plurality of gas phase proto membrane protein ions;
(iv) detaching the one or more residual solvent molecules and one or more non-specifically bound bulk lipid molecules from the intact membrane proteins by irradiating the confined plurality of gas phase proto membrane protein ions with infrared radiation from a laser source to produce a plurality of intact membrane protein ions;
optionally m/z isolating the plurality of gas phase intact membrane protein ions;
(va) optionally subjecting the gas phase intact membrane protein ions to an ion transformative process to produce a plurality of first-generation product ions; Optionally m/z selecting the first-generation product ions;
(vb) m/z analysing and detecting the gas phase intact membrane protein ions or, if step (va) has been performed, the first-generation product ions; and
(vi) utilizing the m/z-ion signal intensity data obtained in step (v) (for instance, when said data is aggregated with data from multiple applications of the method to said multiple copies of the intact protein to provide the m/z spectrum of the product ions of the particular intact membrane protein ion) to obtain information about the structure and/or identity of the intact membrane protein.

Note that if multiple types of first-generation product ions are produced, optionally m/z selecting the first generation product ions may involve m/z selecting one or more types of first-generation product ions (which can be achieved by appropriate m/z selection).

A method of analysing a sample comprising multiple copies of an intact membrane protein, the method comprising:
(i) providing a sample comprising a non-synthetic biological membrane material comprising the multiple copies of the intact membrane protein, a solvent, and bulk lipid molecules, wherein the non-synthetic biological membrane material is extracted from or secreted directly by a cell, an organelle, a viral envelope or a vesicle, and sonicating the sample;
(ii) ionising the sample to produce a plurality of gas phase proto membrane protein ions, each of said gas phase proto membrane protein ions comprising the intact membrane protein non-specifically bound to one or more bulk lipid molecules and to one or more residual solvent molecules;
(iii) confining the plurality of gas phase proto membrane protein ions;
(iv) detaching the one or more residual solvent molecules and one or more non-specifically bound bulk lipid molecules from the intact membrane proteins by irradiating the confined plurality of gas phase proto membrane protein ions with infrared radiation from a laser source to produce a plurality of intact membrane protein ions, and optionally m/z isolating the plurality of gas phase intact membrane protein ions; and
(v) m/z analysing and detecting the plurality of gas phase intact membrane protein ions and/or ions derived therefrom.

A method of analysing a sample comprising an intact membrane protein (especially multiple copies of the intact membrane protein), the method comprising:
(i) providing a sample comprising a non-synthetic biological membrane material comprising the intact membrane protein, a solvent, and bulk lipid molecules, wherein the non-synthetic biological membrane material is extracted from or secreted directly by a cell, an organelle, a viral envelope or a vesicle, and sonicating the sample;
(ii) ionising the sample by electrospray ionisation to produce a gas phase proto membrane protein ion, said gas phase proto membrane protein ion comprising the intact membrane protein which is associated with a submicron-scale membrane fragment or vesicle, and with one or more residual solvent molecules;
(iii) confining the gas phase proto membrane protein ion;
(iv) detaching the one or more residual solvent molecules and one or more non-specifically bound bulk lipid molecules from the intact membrane protein by irradiating the confined gas phase proto membrane protein ion with infrared radiation from a laser source to produce a gas phase intact membrane protein ion, and optionally m/z isolating the gas phase intact membrane protein ion; and
(v) m/z analysing and detecting the gas phase intact membrane protein ion and/or an ion derived therefrom.

A method of analysing a sample comprising multiple copies of an intact membrane protein, the method comprising:
(i) providing a sample comprising a non-synthetic biological membrane material comprising the multiple copies of the intact membrane protein, a solvent, and bulk lipid molecules, wherein the non-synthetic biological membrane material is extracted from or secreted directly by a cell, an organelle, a viral envelope or a vesicle, and sonicating the sample;
(ii) ionising the sample by electrospray ionisation to produce a plurality of gas phase proto membrane protein ions, each of said gas phase proto membrane protein ions comprising the intact membrane protein which is associated with one or more submicron-scale membrane fragments or vesicles, and with one or more residual solvent molecules;
(iii) confining the plurality of gas phase proto membrane protein ions, and exposing the plurality of gas phase proto membrane protein ions to a collision gas to remove one or more residual solvent molecules and one or more non-specifically bound bulk lipid molecules by collision-induced dissociation;
(iv) detaching the one or more residual solvent molecules and one or more non-specifically bound bulk lipid molecules from the intact membrane protein by irradiating the confined plurality of gas phase proto membrane protein ions with infrared radiation from a laser source to produce a plurality of gas phase intact membrane protein ions, and optionally m/z isolating the plurality of gas phase intact membrane protein ions; and
(v) m/z analysing and detecting the plurality of gas phase intact membrane protein ions and/or ions derived therefrom.

A method of analysing a sample comprising multiple copies of an intact membrane protein, the method comprising:
(i) providing a sample comprising a non-synthetic biological membrane material comprising multiple copies of the intact membrane protein, a solvent, and bulk lipid molecules, wherein the non-synthetic biological membrane material is extracted from or secreted directly by a cell, an organelle, a viral envelope or a vesicle, and sonicating the sample;
(ii) ionising the sample by electrospray ionisation to produce a plurality of gas phase proto membrane protein ions, each of said gas phase proto membrane protein ions comprising the intact membrane protein which is associated with one or more submicron-scale membrane fragments or vesicles, and with one or more residual solvent molecules;
(iii) confining the plurality of gas phase proto membrane protein ions, and exposing the plurality of gas phase proto membrane protein ions to a collision gas to remove one or more residual solvent molecules and one or more non-specifically bound bulk lipid molecules by collision-induced dissociation;
(iv) detaching the one or more residual solvent molecules and one or more non-specifically bound bulk lipid molecules from the intact membrane protein by irradiating the confined plurality of gas phase proto membrane protein ions with infrared radiation from a laser source to produce a plurality of gas phase intact membrane protein ions, the laser source having a wavelength of from 1.4 μm to 1 mm, preferably between 9 and 11 μm, and preferably wherein the period of irradiation is less than 50 ms;
optionally m/z isolating the plurality of gas phase intact membrane protein ions; and
(v) m/z analysing and detecting the plurality of gas phase intact membrane protein ions and/or ions therefrom.

A method of analysing a sample comprising multiple copies of an intact membrane protein, the method comprising:
(i) providing a sample comprising a non-synthetic biological membrane material comprising multiple copies of the intact membrane protein, a solvent, and bulk lipid molecules, wherein the non-synthetic biological membrane material is extracted from or secreted directly by a cell, an organelle, a viral envelope or a vesicle, and sonicating the sample;
(ii) ionising the sample by electrospray ionisation to produce a plurality of gas phase proto membrane protein ions, each of said gas phase proto membrane protein ions comprising the intact membrane protein which is associated with one or more submicron-scale membrane fragments or vesicles, and with one or more residual solvent molecules;
(iii) confining the plurality of gas phase proto membrane protein ions, and exposing the plurality of gas phase proto membrane protein ions to a collision gas to remove one or more residual solvent molecules and one or more non-specifically bound bulk lipid molecules by collision-induced dissociation;
(iv) detaching the one or more residual solvent molecules and one or more non-specifically bound bulk lipid molecules from the intact membrane protein by irradiating the confined plurality of gas phase proto membrane protein ions with infrared radiation from a laser source to produce a plurality of gas phase intact membrane protein ions, the laser source having a wavelength of from 1.4 μm to 1 mm, preferably between 9 and 11 μm, and preferably wherein the period of irradiation is less than 50 ms;
optionally m/z isolating the plurality of gas phase intact membrane protein ions;
(va) subjecting the plurality of gas phase intact membrane protein ions to an ion transformative process to produce a plurality of first generation product ions, typically by either collision-induced dissociation or IRMPD;
optionally m/z isolating the plurality of first generation product ions; and either
(vb) detecting and m/z analysing the plurality of first generation product ions, or
(vc) fragmenting the plurality of first generation product ions to produce a plurality of second generation product ions, typically by either collision-induced dissociation or IRMPD;
optionally m/z isolating the plurality of second generation product ions; and
(vd) m/z analysing and detecting the plurality of second generation product ions.

Note that if multiple types of first-generation or second-generation product ions are produced, optionally m/z selecting the first- or second-generation product ions may involve m/z selecting one or more types of first- or second-generation product ions (which can be achieved by appropriate m/z selection).

Any of the methods described herein may additionally comprise:
utilizing the m/z—ion signal intensity data obtained in step (v) (for instance, when said data is aggregated with data from multiple applications of the method on said multiple copies of the intact protein to provide a m/z spectrum of the product ions of the particular intact membrane protein ion) to obtain information about the structure and/or identity of the intact membrane protein.

It is to be understood that although particular embodiments and specific configurations and materials have been discussed herein, various changes or modifications in form and detail may be made without departing from the scope and spirit of this invention. The following examples are provided to illustrate the invention, and should not be considered as limiting.

### EXAMPLES

### Example 1

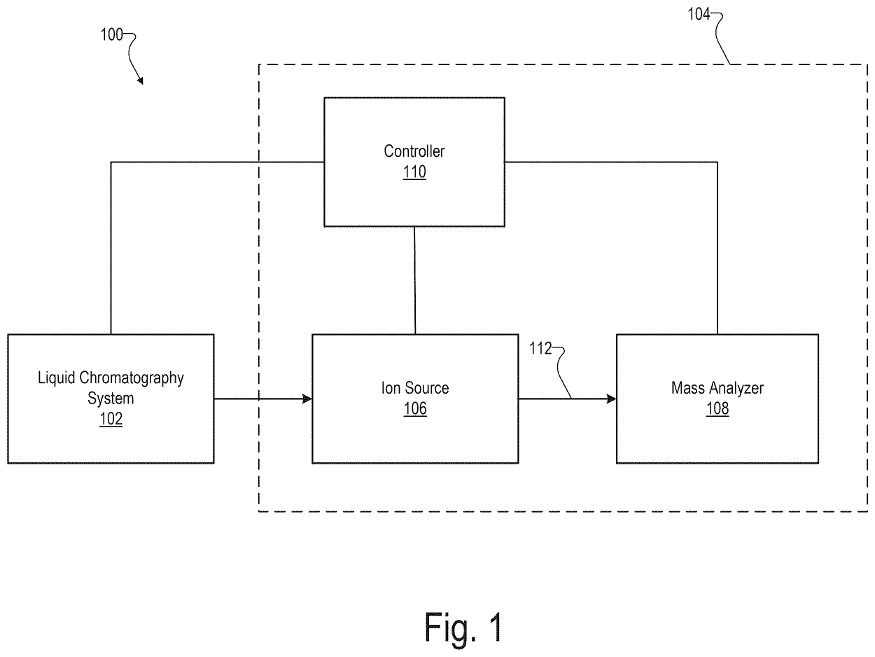

The method has been reduced to practice using a modified Thermo Fisher™ Orbitrap Eclipse™ Tribrid™ mass spectrometer. However, as explained above, any mass spectrometer instrument, with a suitable m/z analyser m/z range that is equipped with an infrared laser light source (10.6 μm) and configured such that an infrared light beam of sufficient intensity may interact with confined proto membrane ions produced from a sample (as described above) by native electrospray ionisation for a sufficient duration to yield fully liberated intact membrane protein ions. Referring now to FIG. 1, which is a simplified schematic depiction of the modified Orbitrap Eclipse Tribrid MS instrument, the following is a description of how various embodiments of the method of the invention are performed with this instrument.

The sample, as generally described above and for specific instances described below, in a native pH solution, is ionised with electrospray ionisation (static native electrospray source) 1 to produce proto membrane protein ions, comprising bulk lipids, solvent molecules, small molecules, as well as other lipids and lipophilic compounds that are specifically associated with membrane proteins and complexes, membrane associated proteins, along with other biomolecules that are associated with membrane bilayers. Said proto membrane protein ions are transferred through a heated metal capillary inlet 2 that represents the atmospheric pressure to vacuum interface of the mass spectrometer. The proto membrane protein exiting the inlet 2 enter a RF ion funnel ion guide 3 and are transmitted through the first vacuum region 21, then traverse the second vacuum region 22 via a first (linear) RF multipole ion guide 4, then traverse a third vacuum region 23 via a second (curved) RF multipole ion guide device 5. The proto membrane protein ions can be optionally subjected to CID within the first RF multipole ion guide 4 by adjusting the voltage gradients along and/or between devices 2, 3, 4, and/or 5 to accelerate the proto membrane protein ion velocities such that collisions with background (preponderantly atmospheric) gas molecules in RF multipole ion guide 4 lead to increases in ion vibrational energy and can induce non-covalent and covalent bond (disadvantageously) dissociation of the proto membrane ions (inlet CID). At suitably chosen accelerating multipole device bias potentials, the covalent bond cleavages from the inlet CID may lead to partial de-solvation and de-lipidisation of the proto membrane protein ions with limited or no loss of non-covalent binding partners or cleavage of covalent bonds and therefore can be advantageous. The proto membrane protein ions exiting the second RF multipole 5 enter and traverse vacuum region 24 via a quadrupole m/z analyser 6, an electrostatic ion gating lens 8, a RF multipole ion guide device 7, an RF C-trap ion transfer device 9 into an RF multipole ion trapping/ion guiding device, the ion routing multipole (IRM) 11. The potentials on the electrostatic ion gating lens 8 can be switched to permit or deflect and eliminate the transmission of the ions further down the apparatus. The IRM has its own enclosure 28 so a flow of Nitrogen can be introduced to it to serve as a damping/collision gas without elevating the pressure in the quadruple m/z filter 6 such that ion transmission though the m/z filter when it is operated in the m/z selecting mode is not impaired. From the IRM, the proto membrane protein ions are transmitted into a fourth vacuum region 28 though a RF multipole ion guide 12 and then are stored within a high pressure trapping region of a dual pressure region RF quadrupole linear ion trap linear ion trap (HPT) 13.

After a sufficient population of proto membrane protein ions are accumulated in the HPT 13, the potentials of the electrostatic ion gating lens 8 are switched to cut off further transmission and accumulation of ions, and the IR laser light source 20 is activated and its output IR light beam 16 is directed down the axis of the HPT 13 to irradiate and vibrationally activate the proto membrane protein ions confined along the devices axis in the central section of the device 13.2. As is well known in the art, the DC bias potentials of the of the front section 13.1, the centre section 13.2 and back section 13.3 of the HPT create an axial confinement potential and the radial confinement is provided by the RF quadrupole field established by the RF potentials applied to the device's electrodes. The elevated pressure of the Helium (˜6 mTorr) within the HPT enables collisional stabilisation and accumulation of the proto membrane protein ions injected into the HPT and the localization of the proto membrane protein ions along the device's axis. An IR transparent optical window 19 enables the laser beam to enter the vacuum chamber at the back end of the ion trap vacuum region 27. The beam from IR laser source has previously been aligned with the axis of the HPT using mirrors 17, and the IR lenses 18 creates an elongated waist in the IR light beam along the HPT 13 axis in the centre section 13.1 of the HPT. The residual solvent and bulk lipid molecules non-covalently bound to the proto membrane protein ions absorb the photons from the IR beam 16 which vibrationally excites them. This causes dissociation of all solvent and bulk lipids from a significant portion of the confined proto membrane protein ions to yield a population of intact membrane protein ions which remain trapped within the HPT 13. In some instances, prior to the irradiation of the proto membrane protein ions by the IR beam, it is advantageous to apply a wide m/z isolation band auxiliary voltage waveform to the HPT 13 in order to eliminate ions from the device that have m/z values that are outside the m/z range of most of the accumulated proto membrane ions in order to eliminate various background ions that are co-produced and transmitted with the proto-membrane ions.

After the cessation of IR activation of the ions (proto membrane protein ions, liberated intact membrane protein ions, residual background ions etc.) confined in the HPT, this population of the ions can be analysed according to the methods established in the art for the Orbitrap Eclipse Tribrid instrument. This population of ion can be analysed as one would treat a population of ions by generated native electrospray ionisation from an “ordinary” sample comprised of soluble proteins and protein complexes, transmitted into the instrument, de-solvated by inlet CID and delivered to and confined in the HPT 13. For example, m/z analysis of the post IR irradiation ion population in the HPT may be achieved by transfer to that ion population to the C-Trap 9 from which the ion population is ejected radially though optical elements in vacuum region 26 and into the Orbitrap m/z analyser 10 in the ultra high vacuum region 25. The Orbitrap m/z spectrum of this population of ions (generally obtained through averaging the detected ion signal transient data from many iterations of the experiment and therefore m/z analysis of multiple populations of ions) would comprise m/z peaks corresponding various charge states of liberated intact membrane protein ions from pluralities of particular membrane protein isoforms in the sample as well as m/z peaks and elevated baseline corresponding to various other background ion types including residual PMP ions. Alternatively, the ion population in the HPT 13 may be transferred to the low pressure trapping region of the dual pressure region RF quadrupole linear ion trap linear ion trap (LPT) 14, and m/z analysed by m/z sequential radial ejection and detected by electron multiplier based ion detector 15. The ion trap m/z spectrum of this population of ions (generally the result of averaging the m/z and detected ion signal data from many iterations of the experiment and therefore m/z analysis of multiple populations of ions) would comprise m/z peaks corresponding to various charge states of liberated intact membrane protein ions from pluralities of particular membrane protein isoforms in the sample as well as m/z peaks and elevated baseline from various other background ion types including residual PMP ion.

To perform tandem mass spectrometry (MS/MS, or MS²) analysis on the liberated intact membrane protein ions released from PMP ions by the IR irradiation of the ions in the HPT 13, a narrow m/z range of ions is isolated in the ion trap by applying a narrow m/z isolation band auxiliary voltage waveform to the HPT 13 in order to eliminate ions from the device that have m/z values that are outside of a narrow corresponding to the m/z of a particular charge state of a membrane protein ion of interest as determined by m/z peaks observed in a previously acquired m/z spectrum. After application of this m/z isolation waveform, the population of ions remaining in the HPT are a plurality of intact membrane protein ions of the particular charge state of interest of the chosen intact membrane protein ions and lessor numbers of background ions of various types as well as residual PMP ions and, potentially, intact membrane protein ions from other protein isoforms present in the sample at much lower abundance than the protein isoform under interrogation by MS/MS. The m/z isolated ions are then subjected to one or more of the various ion transformative processes available on the instrument. The m/z isolated ions can be transferred to the IRM 7 at relatively high energies to perform beam-type HCD. Or the m/z isolated ions can be retained in the HPT 13 and subjected ion trap type (resonant) CID, IRMPD, ETD, AI-ETD or PTCR. [Note: Trap type (resonant) CID and ETD and PTCR are standard ion transformational processes available on commercially available configurations Thermo Fisher™ Orbitrap Eclipse™ Tribrid™ mass spectrometer and the manner of how these processes are effected on the instrument is well known in the art. However the most significant modification of the instrument and the one that was needed to demonstrate the method of the invention was the adaptation of the IR laser light source 20 to the instrument so as to enable IR irradiation of ions confined in the HPT 13 (and LPT 14). This of course, enables, as described above, the irradiation of the PMP ions to effect full “evaporation’ of solvent and bulk lipids from these ions to yield IMP ions. Advantageously this modification also enables IRMPD and AI-ETD on the instrument.] To effect IRMPD on the m/z isolated population ions in the HPT 13, substantially comprising IMP ions, the IR laser 20 is activated for a time interval an output power above that appropriate for the efficient conversion of PMP ions to IMP ions without inducing covalent bond cleavage. To effect AI-ETD the IR laser light source is activated while isolated ions are subjected to ion=−ion reaction with ETD reagent ions in HPT 13. The IR laser light source output power levels for AI-ETD are similar to those used for the conversion of PMP ions to IMP ions according to the invention. The product ions of any of these transformation processes can be then delivered to the Orbitrap analyser 25 or the LPT 14 for m/z analysis and detection as described in above in the first example describing MS analysis the IMP ions generated through IR irradiation of PMP ions in the HPT 13.

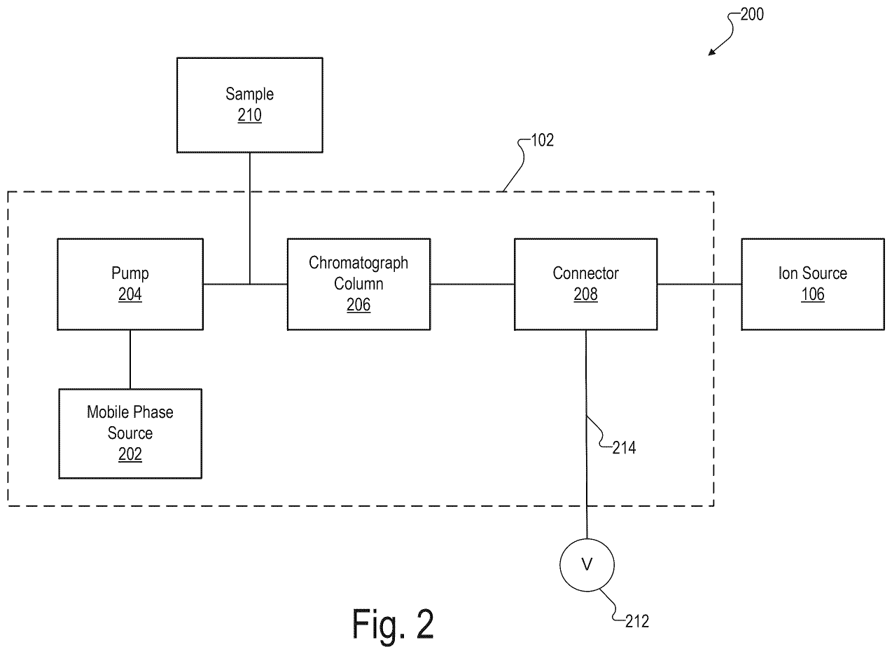

In the following discussion of FIGS. 2-7, which demonstrate the utility of the methods according to the invention, the process of full removal of solvent molecules and bulk lipid molecules from PMP ions through IR irradiation induced vibrational activation to produce IMP ions is, for purposes of brevity, referred to as IRMPD-L. IRMPD-L stands for InfraRed Multi Photon De-Lipidisation.

Referring now to FIG. 2, the utility of IRMPD-L was demonstrated using vesicles derived from E. coli cells, which were engineered to contain, amongst many proteins endogenous to the inner and outer membranes, the MacAB efflux pump. Membrane fragments were produced using gentle sonication, and the resulting membrane fragments were analysed as described above (e.g., via nano-electrospray ionisation to create PMP ions were transported into the modified Thermo Scientific Orbitrap Eclipse mass spectrometer and delivered to the HPT 13 to produce the intact membrane protein and complex ions, IMP ions, by IRMPD-L). Low IR energies (1.0% of 60 W, viz. 0.6 W) and at low irradiation times (10 ms) produced a poorly resolved charge state m/z peak series (FIG. 2) in the m/z spectrum (averaged) produced by m/z analysis with the Orbitrap analyser of the ions retained in the HPT after the IRMPD-L step. Increasing the laser intensity to 5.0% (˜3.0 W) produced three well-resolved m/z peak series corresponding to consecutive charge states of IMP ions of a common membrane protein isoform (charge state envelope) in the corresponding (averaged) m/z spectrum of the ions retained in the HPT 13 after IRMPD. L step. The most abundant of which was centred at ˜6500 Th) at has 25+ charge state and corresponds in mass to an endogenous membrane protein having a mass of 149,577 Da. Another charge state envelope, centred at the 19+ charge state (˜5500 Th), corresponds to a 104,775 Da protein. Further, a series of low abundances m/z peaks, centred at the 25+ charge state near ˜9000 Th are observed and correspond to a 218,312 Da protein. The most intense peak has a total ion intensity of 2.28×10³. At increased IRMPD-L irradiation energies (7%, ˜4.2 W) the abundance of this peak decreased to 1.66×10³, and charge state distribution corresponding to the 218, 312 Da protein disappeared presumably due to dissociation of the complex into subcomplexes or covalent bond dissociation fragment ions. Increasing the laser power to 9% (ca. 5.4 W) led to further decreases IMP m/z peak intensity, presumably due to the absorption of the IR beam energy by the membrane protein and complex ions released from PMP ions, which causes them to dissociate via IRMPD processes. Thus, when using the IRMPD-L process, the method of the invention, the intensity of the IR irradiation of the PMP ions must be adjusted to maximize the yield IMP ions.

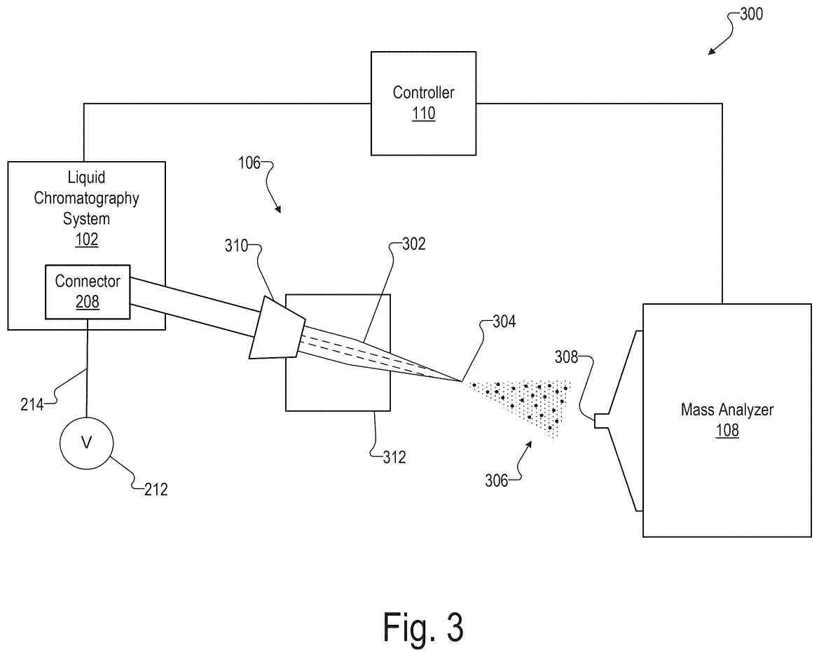

For purposes of comparison, the yield of IMP ions using the generally preferred prior art method for liberation of IMP ions from PMP ions, inlet CID, (now referring to FIG. 1) where the PMP ions are subjected to CID within the first RF multipole ion guide 4 by adjusting the voltage gradients along and/or between RF ion guiding devices 2, 3, 4, and/or 5 was characterised using the same MacAB sample as above. FIG. 3 illustrates the m/z spectra of the ions produced via inlet CID for several collision (accelerating) voltages with the potentials applied adjacent optical elements adjusted optimise ion transmission. At a collision voltage of 50 V, very low ion signal (m/z peak) intensities were observed, but m/z several peaks corresponding to charge states of the 149,577 Da protein were observed. At an increased acceleration voltage of 100V, two charge state series m/z peak envelopes were observed in the m/z spectrum; one which corresponded to the 149,577 Da protein (1.02×10³ intensity on the 25+ ion). The other charge state distribution corresponded to trace amounts of the 102,755 Da protein (19+ centre charge state, near 5000 Th). Other m/z peaks were observed, however, none of which corresponded to a continuous series of charge states (e.g., peaks that have m/z spacing consistent with integer differences in charge state for a common neutral mass). Increasing the acceleration potential to 150 V led to a slight increase in ion intensity (the m/z peak of for 25+ charge state ion of the 149,5777 Da protein increased to 1.26×10³), but the m/z peaks from the 102,755 Da protein disappeared. Further increases in acceleration energy were explored, all of which led to decreased ion signal in the m/z spectrum. Thus, it is clear that for the MacAB sample the IRMPD-L technique was superior to the collision-based technique in removing lipids from the PMP ions as it produced higher m/z peak intensities for m/z peaks corresponding to the intact membrane protein ions (IMP ions) and generated clear m/z spectra which are amenable to peak assignment.

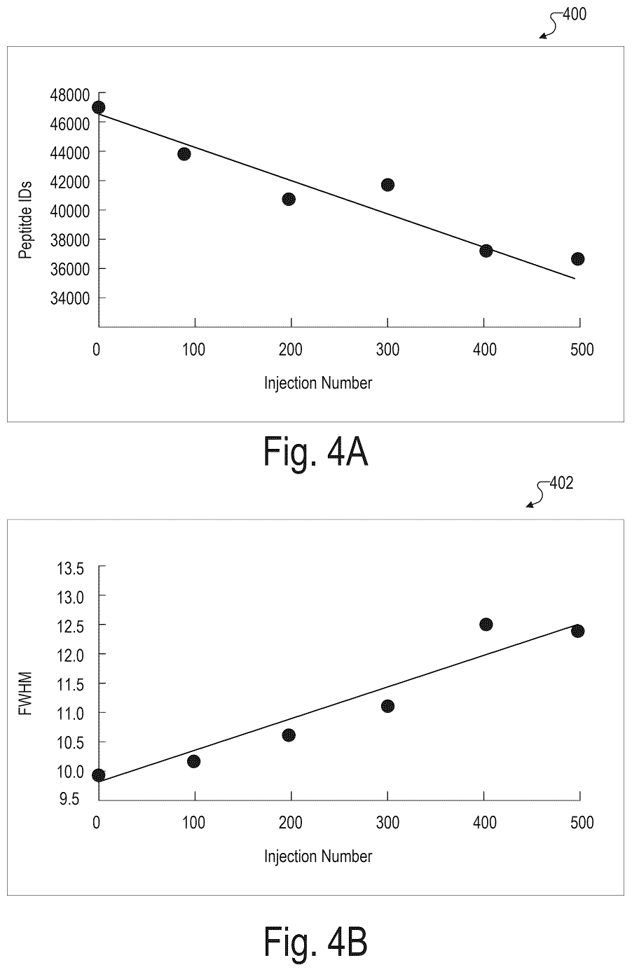

In FIG. 4 the enhanced ability to fully remove non-specifically associated lipid molecules from proto membrane protein ions with IRMPD-L as compared to collision-based approach is further illustrated by the comparing the m/z peaks observed for the 102,775 Da protein (20+ and 10+ charge state) in the m/z spectra acquired with optimised de-lipidation parameters for both the inlet CID (In-source CID) and IRMPD-L. Using IRMPD-L, the higher signal-to-noise ratio of the m/z peaks in the m/z spectrum allows for the clear assignment of charge states these peaks, and observation with the “satellite” m/z peaks, one of which corresponds to adduction of a 129 Da molecule (a metabolite, post-translational modification, or other small molecule ligand to the protein. These m/z peaks are not observed by removing non-specifically bound bulk lipids from the PMP ions using in-source CID). Therefore, FIG. 4 exemplifies the advantage of IRMPD-L over the prior employed collision-based approaches in its ability to produce m/z spectra of membrane protein ions that correspond to the retention of non-covalent (e.g., binding of ligands such as metabolites, small molecules) and/or covalent adducts (e.g., retention of PTMs that are labile and are known to dissociate during CID processes).

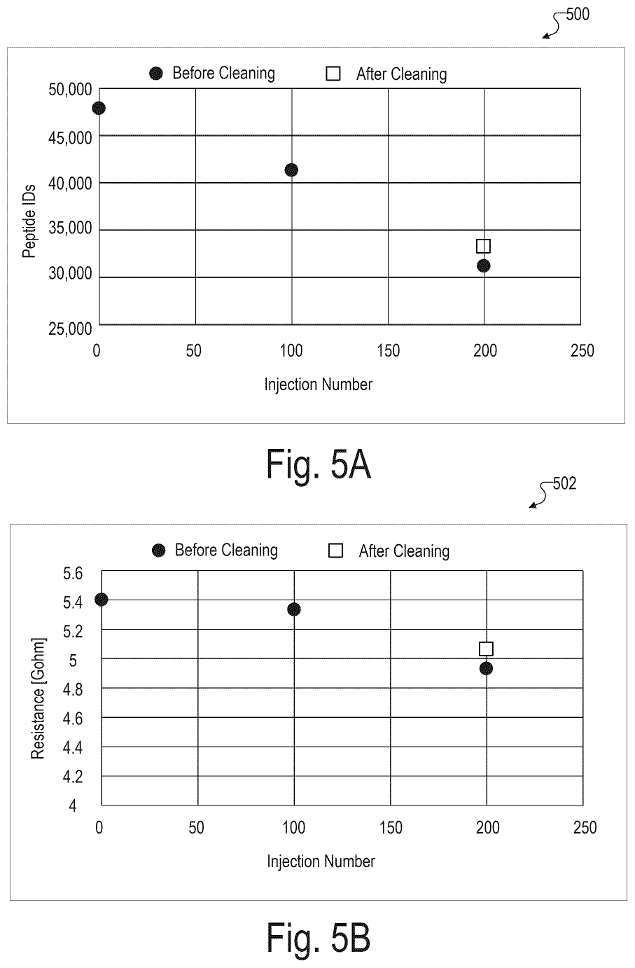

The utility of the IRMPD-L method of the invention to remove solvent and bulk lipid molecules from proto membrane protein ions (PMP ions) comprising multi subunit membrane protein complexes that were originally embedded in vesicles to yield intact membrane protein ions. The heteropentameric β-barrel assembly machinery complex in E. coli was expressed in a cell, and, after harvesting the cell membranes, the membranes were fragmented into smaller membrane vesicles using mild sonication in ammonium acetate buffer. Referring now to FIG. 5, PMP ions were produced by nanoelectrospray ionisation, and membrane protein and complex ions (IMP ions), were produced by de-lipidation of the bulk carrier lipid molecules using the IRMPD-L method of the invention. Using 10 ms IR irradiation interval with 7% of available laser out power setting during the IRMPD-L a m/z spectrum (averaged) was acquired for the ions retained in the HPT and the shown in the upper m/z spectrum shown in FIG. 5. Several m/z peak series corresponding to sequential charge states of membrane protein isoforms were identified, one of these series of m/z peaks (charge state envelopes) corresponded to a 199,200 Da membrane protein complex (centered at the 28+ charge state m/z peak near 7000 Th in FIG. 5), which when converted to a neutral mass has a ˜0.5% deviation from the mass calculated from the known sequence of the overexpressed and fully assembled Bam complex (200,035 Da). The intensities of the m/z peaks for the various charge states of IMP ions (circles in FIG. 5) for this protein decreased as the laser output power setting was increased during IRMPD-L (from 7% to 10% to 15% of maximum output); concomitant with this decrease the abundance of the m/z peaks corresponding to different ionization states of the intact Bam membrane complex was the increase in the intensity of the lower m/z features (m/z peaks), which, presumably, corresponds to increased production of fragment ions including subcomplex ions produced by IRMPD of the intact membrane protein (complex) ions. As can be seen, the upper m/z spectrum contains clear, well-defined m/z peaks and was generated at a laser output power well-below the threshold for standard IRMPD. The m/z peaks have satisfactory intensities.

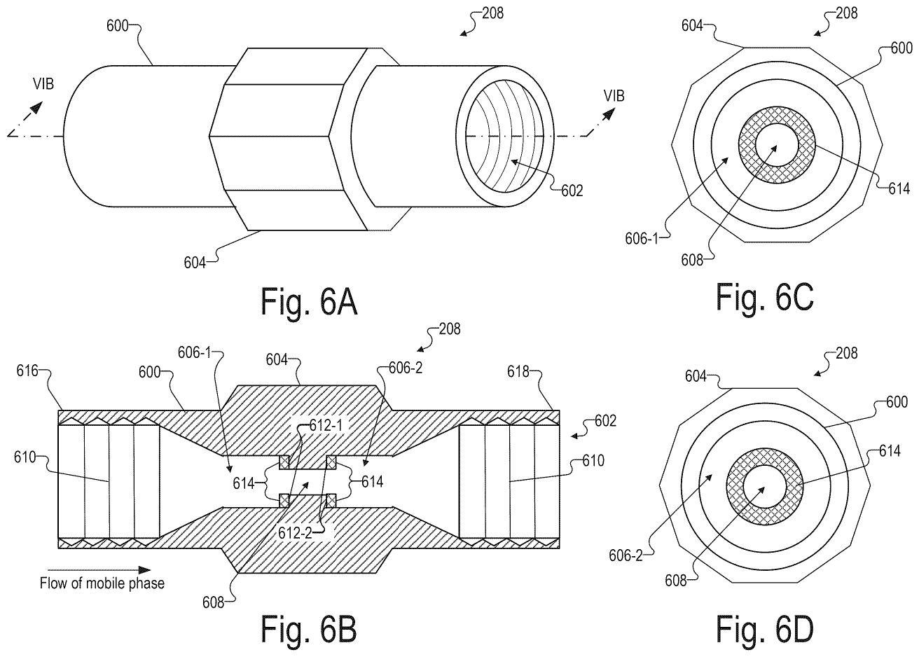

To illustrate that sequence information can be obtained from IMP ions of the fully assembled Bam complex could be obtained, the IMP ions at 7378 Th (the 27+ charge state of the 199 kDa signal distribution) were m/z isolated using a ˜10 Th selection window. Subsequently, this population of IMP ions were subjected to IR multiphoton dissociation (IRMPD). This involved high laser energy (>15% of available energy) to induce cleavage of the peptide bonds of each protein subunit to generate a product ion distribution comprised of peptide and protein fragment ions. The resulting spectrum exhibited many low charge state ions (1+ to 7+) indicating that the majority of the protein complex had been fragmented into amino acid sequence ions. Manually searching this spectrum for expected fragments of all five subunits generated many potential matches, so the experiment focused on characterising two expected ions corresponding to cleavage along the peptide backbone of the small His₆-tagged BamE subunit. A series of m/z peaks with 1 Da spacing corresponding to the singly charged b₂₀ ion were observed (FIG. 6B), along with a well-resolved series of peaks corresponding to the singly charged y₂₇ ion. Importantly, the matched fragment ions contained the engineered C-terminal His₆ tag (FIG. 6C) and were across regions that are not expected to bear any post-translational modifications. Furthermore, comparison of the observed and theoretical masses of the observed fragments showed that the mass deviations were well-within the accepted threshold for confident molecular identification (<10 ppm), providing increased confidence in the assignments.

Therefore, it was shown that it is possible to liberate IMP ions from PMP ions with a laser power of 7% and then, using a higher laser power >15%, to dissociate the intact membrane protein complex to obtain sequence ions that define a subunit released directly from this complex.

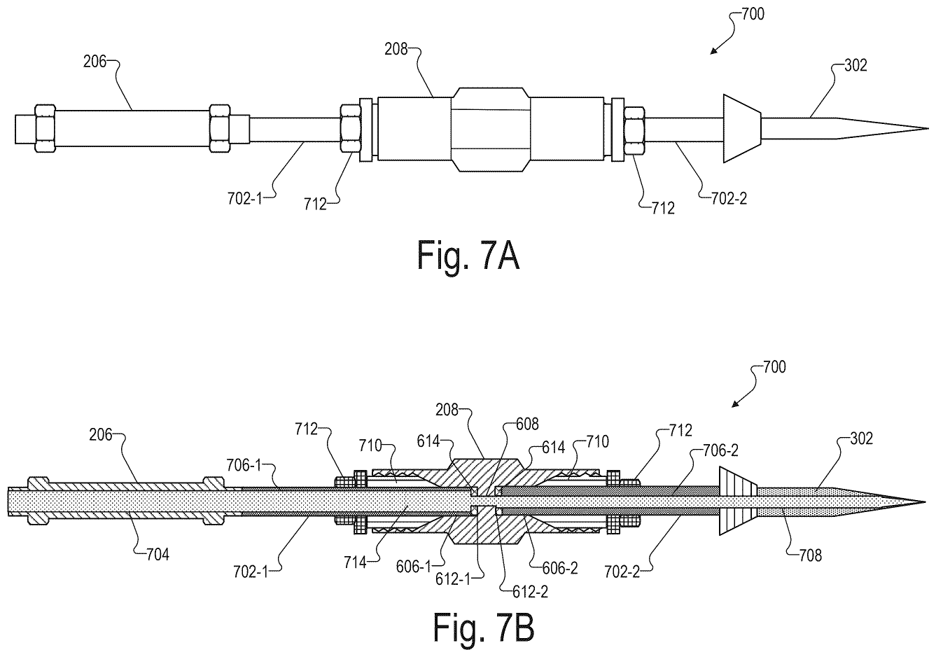

As a final simple illustration of the utility of the IRMPD-L method, FIG. 7 demonstrates that, once optimised parameters are obtained, intact membrane proteins and membrane associated proteins can be liberated from PMP ions comprising entire secreted extracellular vesicles (exosomes) obtained from human fluids.

### Example 2

Experiments were conducted to show the impact of IR laser output power on the liberation of membrane proteins from a native lipid bilayer for detection by mass spectrometry, and application of the method of the invention to the sequencing of rhodopsin following its liberation from the lipid bilayer.

### (a) Methods

### (i) Preparation of Rod Disc Membranes

Bovine eyes were obtained from a commercial slaughterhouse. Rod outer segment membranes were obtained from a batch of 50-100 eyes with dark-adapted retinas, and isolated as previously described.^{[1,2]} Isolated membranes were suspended in 200 mM ammonium acetate and homogenized using a probe sonicator with a stepped tip microtip (2 mm; Vibra-Cell VCX-500 Watt, Sonics) and a maximal amplitude (40%) (1 s on, 2 s off) applying 2 J per cycle for 1.5 min. This resulted in disc membrane vesicles containing rhodopsin at approximately 9 μM and were diluted 2× with 200 mM ammonium acetate buffer at pH 7.4 immediately prior to native MS analysis.

### (ii) Native Mass Spectrometry and Top-Down Mass Spectrometry

Experiments were conducted on a modified Orbitrap Eclipse Tribrid mass spectrometer (Thermo Fisher Scientific). A Synrad Firestar Ti60 CO₂ continuous wave IR laser (10.6 μm, 60 W) was coupled to the instrument such that the beam was focused into the high-pressure cell of the dual cell quadrupole linear ion trap (QLIT) and its timing and power output were controlled by the instrument software. These instrument modifications have been described previously.^{[3]} Vesicles were ionized via nanoelectrospray using a gold-coated borosilicate glass capillaries (1.2 mm O.D.) prepared in house. The capillary voltage was held at 1.0 to 1.2 kV relative to the instrument orifice (heated to ˜100-200° C.). Ions were activated in the source using gentle activation energy (50 V source CID) before entering the next differential pressure region containing the bent flatapole. The instrument was operated in high-pressure mode (20 mtorr in the ion routing multipole), and ions were irradiated in the ion trap for 25 ms at laser output powers ranging from 0 to 6 W (0-10% output power) before being transferred back into the orbitrap for MS¹ detection. MS¹ spectra were collected at a resolution of 15,000 @ m/z 200. For MS² experiments, ions were isolated in the ion trap and irradiated with the IR laser for 5 or 10 ms at laser output powers ranging from 9.0 to 10.8 W (15-18%) to induce peptide bond dissociation. Fragment ions were then back transferred into the Orbitrap for detection at a resolution of 200,000 @ m/z 200.

(iii) Data Analysis and Software

Proteins were first identified through a database search of the entire bovine proteome using Prosight Native (Proteinaceous).^{[4]} Fragments were then manually validated using TDValidator (Proteinaceous).^{[5]} Assigned peaks were manually inspected and incorrect assignments were removed.

### (b) Results

(i) Liberating Membrane and Peripheral Proteins from Vesicles by IR Light

We previously described a mass spectrometer which incorporated a 10.6 μm wavelength CO₂ laser directed into the high pressure cell of a linear ion trap, and demonstrated that the laser output power and irradiation time could be modulated.^{Error! Bookmark not defined.}

Here, we sought to determine whether this method was capable of liberating endogenous membrane proteins embedded within vesicles derived from their native lipid bilayer. This was not previously considered achievable, as such proteins are expected to have a relatively low absorption cross-section at the wavelength of light used here.

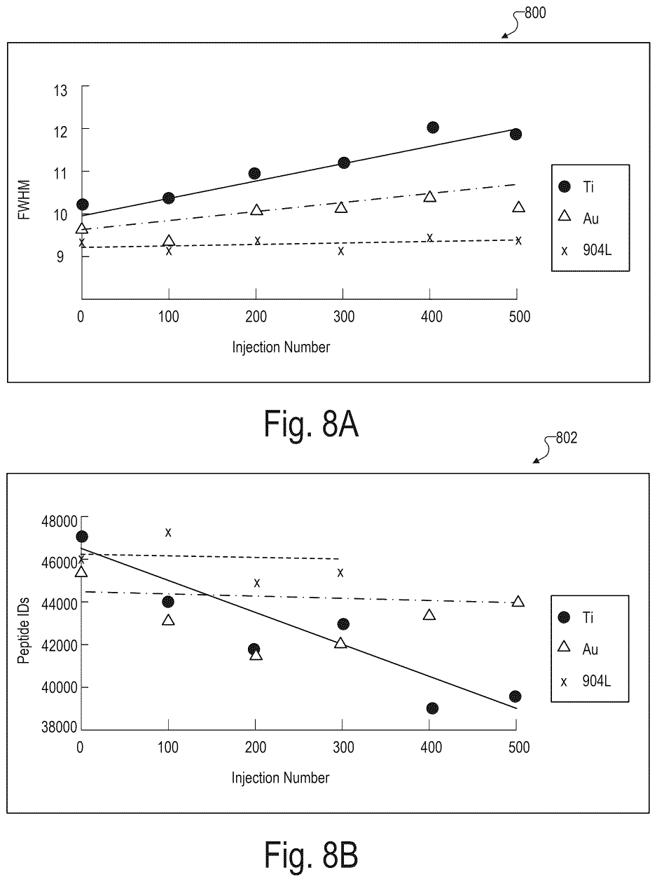

To explore the impact of laser output power on the lipid vesicles, the output power was increased in 0.6 W increments (1% of the total laser output power) while maintaining a constant irradiation time of 25 milliseconds (FIG. 8). The resultant mass spectra at each laser output power look different. At a low laser output power of 1.8 W, few discernible peaks corresponding to 44448, 45619, 47194, and 51705 Da emerge from the apparent high baseline. The high baseline can be attributed to the inherent heterogeneity of the vesicle; ion signals are detected at every m/z, ultimately precluding the charge state determination of lower abundance protein species. The mass spectrum which is produced following irradiation with a laser output power of 5.4 W instead reveals well-resolved peaks for a number of protein distributions, including new charge state distributions not observed under previous conditions. The most abundant distribution >4500 m/z is tentatively assigned to a GPCR, rhodopsin, and corresponds to a molecular weight of 41714 Da with additional satellite features, indicative of the presence of several proteoforms of that protein species which co-exist in disc membranes.

The marked difference in observed protein distributions at each laser output power suggest that the IR laser can affect differential release of proteins from lipid vesicles. Without being bound by theory, we hypothesize that at relatively low laser output powers (0.6 to 4.2 W), the laser irradiation fractures the lipid bilayer, releasing soluble proteins trapped within the core of the vesicles. At higher laser output powers, we hypothesize that the absorption cross-section of bulk phospholipids is sufficiently high such that the lipid bilayer dissipates, thus liberating membrane proteins and related complexes for mass spectrometry analysis. By careful tuning of these parameters, many non-covalent protein-protein interactions remain unperturbed.

### (ii) Native Top-Down Sequencing of Rhodopsin Proteoforms

The high-resolution mass spectrum obtained using a laser output power of 5.4 W reveals heterogeneity in the rhodopsin proteoforms, as there are six observed distributions between m/z 5160-5300 which correspond to an 8+ charge state. The deconvoluted masses of the main peak of each major proteoform correspond to 41476 Da, 41714 Da, 41876 Da, 42038 Da, 42200 Da, and 42362 Da. Each proteoform significantly deviates from the reported sequence mass of 39008 Da, and therefore we attribute the additional masses (of 2468 up to 3354 Da) to the various post-translational modifications that have been reported. Rhodopsin is the most well-characterized GPCR and various post-translational modifications have been reported: acetylation of M1, N-linked glycosylation at N2 and N15, palmitoylation at S322 and S323, phosphorylation at seven potential sites (S334, T335, T336, S338, T340, T342, S343), and a disulfide bond between C110 and C187 which links the intradiscal loops 1 and 2. Using the intact mass, we can infer which modifications may be present. For example, the difference in mass between the two lowest molecular weight proteoforms (41476 Da and 41714 Da) is 238 Da, potentially indicating a difference in palmitoylation at one of the two sites prone to lipidation. Furthermore, the mass differences between the latter five proteoforms is consistently 162 Da, suggesting heterogeneity in glycan arrangement through the subsequent addition of single hexoses. The identity of the glycans and their occupancies were confirmed using glycoproteomics, and the abundances were then used to assign the observed proteoforms in the native mass spectrum. The proteoform with the highest abundance in the native mass spectrum therefore contains two N-linked glycans at sites N2 and N15, and the glycans are each site are both comprised of Man₃GlcNAc₃. For the remaining proteoforms, the N-linked glycan at site N2 remains static, while the glycan at N15 can differ by a single mannose, yielding proteoforms with Man₄GlcNAc₃, Man₅GlcNAc₃, and Man₆GlcNAc₃ at N15. However, the remaining mass difference between the sequence mass and the measured mass (>500 Da) is less straightforward to assign, as the mass can be realized using combination of several other post-translational modifications.

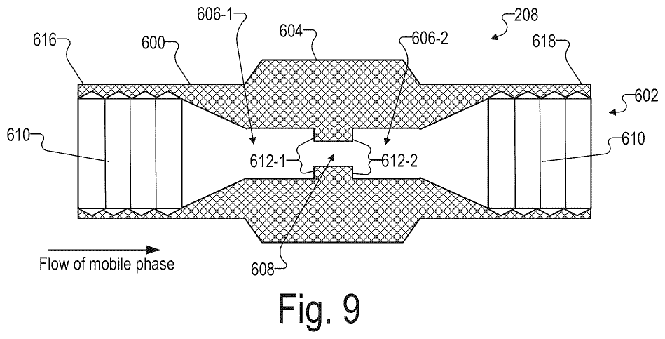

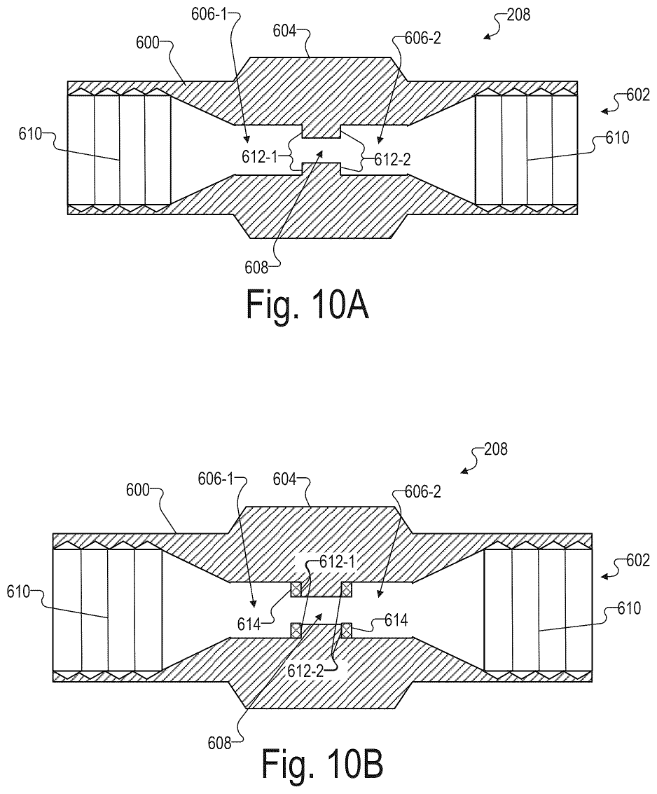

To confirm the identity and localization of specific post-translational modifications, we isolated the 8+ charge state at m/z 5215 using an m/z window of 35 Da. The isolation window therefore encompassed any proteoforms present within the mass range of ˜41.5 to 41.9 kDa, which were then subjected to infrared multiphoton dissociation (IRMPD) using short (5 ms) irradiation times of high laser output powers (15-18% or 9-10.8 W) to generate sequence-informative ions from the intact membrane protein. The most abundant fragments in the MS² spectrum are assigned to y-type fragment ions (y₁₀¹⁺-y₂₂¹⁺) which originate from backbone cleavages within the highly flexible C-terminus (FIG. 9). The good C-terminal sequence coverage reveals evidence for a number of key post-translational modifications, and the sequential fragmentation of adjacent amino acids (i.e. sequence tags) allows for their precise localization. There is ample information embedded within the series of sequential fragments from y₁₀¹⁺-y₂₂¹⁺; we find evidence for both unmodified and phosphorylated fragment ions for y₁₅₋₁₉¹⁺ which localize the phosphate modification to S334. The highly abundant 3+ and 2+ fragment series above 2500 m/z contain even more information; the cluster of triply charged fragment ions between 2540 and 2620 m/z can be attributed to y-type fragments y₆₆³⁺, y₆₇³⁺, and y₆₈³⁺ which originate from cleavages within the intradiscal loop 3. Fragments which originate from the same backbone cleavages in intradiscal loop 3 are also present as doubly charged ions between m/z 3800 and 3900, where the relative distribution of one and two palmitate modifications are maintained.

Despite the prevalence of several sequence tags in the fragmentation data which aid in the confident assignment of rhodopsin, the presence of N-linked glycans at N2 and N15 precludes analysis of N-terminal fragments using simple arithmetic to determine amino acid mass differences between fragment ions. Over 300 fragment ions are observed in the MS² spectrum, which is a notable difference from the approximately 100 to 150 fragment ions detected for other proteins in the vesicles. This is attributed to the greater number of fragment ions that can be generated from a single backbone cleavage, as concomitant glycan fragmentation will combinatorially increase the number of observed b-type fragment ions. For proton-driven fragmentation, such as in IRMPD, glycans fragment predictably along glycosidic bonds, producing Y and Z-type fragments, and across the glycan ring, resulting in X-type fragments. The masses of these theoretical fragments can then be appended to the theoretical masses of the protein fragment ions which originate from amino acid cleavages that encompass the N-linked glycan. We searched for fragment ions which would result from every possible combination of glycan and protein cleavages and found evidence for fragments which originate from backbone cleavages at the N-terminus of rhodopsin. In many cases, several assigned fragments originated from the same protein backbone cleavage, but retained differences in partial glycans covalently linked to asparagine 2 and 15. Under the dissociation conditions used here, the partial glycans which remain are typically only comprised of the N-acetylglucosamine core, and few fragments could be assigned which retained any part of the branched mannose moiety. This is expected as collision-based and IR-based fragmentation typically do not preserve the fragile branching of glycan modifications. Despite this, the goal was not to reconstruct the complex glycan from the native fragmentation spectrum; instead we identified patterns in the fragment ions such that the sequence information of heterogeneous membrane proteins could be obtained, unobstructed. Altogether, we achieve 14% sequence coverage on an endogenous GPCR, directly from its native lipid bilayer, including direct evidence and localization of several key PTMs such as phosphorylation, palmitoylation, and N-linked glycosylation. Further analysis would allow still higher sequence coverage.

Thus, the methods disclosed herein provide a robust approach for accurately characterising intact membrane proteins in a native environment. Whilst this example describes the characterisation of rhodopsin as an illustrative protein, the disclosed methods are amenable to a wide variety of membrane protein analytes.

### REFERENCES

1. Jastrzebska et al. (2006) “Functional and Structural Characterization of Rhodopsin Oligomers”, Mechanisms of Signal Transduction, Vol. 281, pp. 11917-11922
2. Kevany et al. (2013) “Structural and Functional Analysis of the Native Peripherin-ROM1 Complex Isolated from Photoreceptor Cells”, Membrane Biology, Vol. 288, pp. 36272-36284
3. Lutomski et al. (2023), “Infrared Multiphoton Dissociation Enables Top-Down Characterization of Membrane Protein Complexes and G Protein-Coupled Receptors”, Angew. Chem. Int. Ed., Vol. 62, e2023056
4. Durbin et al. (2023), “ProSight Native: Defining Protein Complex Composition from Native Top-Down Mass Spectrometry Data”, J. Proteome Res., Vol. 22, pp. 2660-2668
5. Fornelli et al. (2018), “Accurate Sequence Analysis of a Monoclonal Antibody by Top-Down and Middle-Down Orbitrap Mass Spectrometry Applying Multiple Ion Activation Techniques”, Anal. Chem., Vol. 90, pp. 8421-8429

1. A method of analysing a sample comprising an intact membrane protein, the method comprising:
(i) providing a sample comprising a non-synthetic biological membrane material comprising the intact membrane protein, a solvent, and bulk lipid molecules;
(ii) ionising the sample to produce a gas phase proto membrane protein ion, said gas phase proto membrane protein ion comprising the intact membrane protein non-specifically bound to one or more bulk lipid molecules and to one or more residual solvent molecules;
(iii) confining the gas phase proto membrane protein ion;
(iv) detaching the one or more residual solvent molecules and one or more non-specifically bound bulk lipid molecules from the intact membrane protein by irradiating the confined gas phase proto membrane protein ion with infrared radiation from a laser source to produce a gas phase intact membrane protein ion; and
(v) m/z analysing and detecting the gas phase intact membrane protein ion and/or an ion derived therefrom.

2. The method of claim 1, wherein the sample comprises a non-synthetic biological membrane material extracted from or secreted directly by a cell, an organelle, a viral envelope or a vesicle.

3. The method of claim 1 or claim 2 wherein the gas phase intact membrane protein ion is free of non-specifically bound bulk lipid molecules, and is preferably also free of other, non-lipid, non-specifically bound molecules.

4. The method of any preceding claim wherein, in the non-synthetic biological membrane material provided in step (i), the intact membrane protein is present in or attached to a lipid bilayer comprising the bulk lipid molecules.

5. The method of claim 4 wherein the lipid bilayer forms a vesicle.

6. The method of any preceding claim wherein step (i) comprises sonicating the sample.

7. The method of any preceding claim wherein the sample is ionised by electrospray ionisation, preferably by nanoelectrospray ionisation or by static electrospray ionisation.

8. The method of any preceding claim wherein the sample is ionised by native electrospray ionisation.

9. The method of any preceding claim wherein the gas phase proto membrane protein ion of step (ii) comprises one or more submicron-scale membrane fragments or vesicles associated with the intact membrane protein and with solvent molecules.

10. The method of any preceding claim which additionally comprises exposing the gas phase proto membrane protein ion to a collision gas to remove one or more residual solvent molecules by collision-induced dissociation.

11. The method of any preceding claim wherein a wavelength of the infrared radiation from the laser source preferentially excites one or more vibrational modes of one or more bonds within the non-specifically bound bulk lipid molecules.

12. The method of any preceding claim wherein the infrared radiation from the laser is continuous wave radiation.

13. The method of any preceding claim wherein the wavelength of the infrared radiation from the laser source is from 780 nm to 1 mm, preferably from 1.4 μm to 1 mm, more preferably from 3 μm to 100 μm, most preferably between 9 and 11 μm.

14. The method of any preceding claim, wherein the wavelength of the infrared radiation from the laser source has a principal wavelength at one or more of 10.6 μm, 10.2 μm, 9.6 μm, and 9.3 μm.

15. The method of any preceding claim wherein the irradiation with the infrared radiation is performed for a period of up to 1 second, preferably for a period of less than 50 ms.

16. The method of any preceding claim wherein step (v) comprises:
(va) subjecting the gas phase intact membrane protein ion to an ion transformative process to produce a first-generation product ion; and
(vb) m/z selecting the first-generation product ion.

17. The method of claim 16 which comprises:
(vc) subjecting the first-generation product ion to a further ion transformative process to produce a second generation product ion; and
(vd) m/z analysing and detecting the second generation product ion.

18. The method of claim 16 or claim 17 wherein each ion transformative process is selected from infrared multiphoton dissociation, electron transfer dissociation, activated ion electron transfer dissociation, electron capture dissociation, UV photodissociation and collision-induced dissociation.

19. The method according to any of claims 16 to 18 which further comprises
(vi) analysing the m/z spectral data of the product ion to obtain information about the structure and/or identity of the intact membrane protein.

20. The process according to claim 19 wherein the information includes the amino acid sequence of all or part of the intact membrane protein, and/or the location and/or type of a post-translational modification of the intact membrane protein.

21. The method according to any preceding claim wherein the gas phase proto membrane protein ion and/or the gas phase intact membrane protein ion is held in an ion trap or transmitted through an ion guide or mass filter, and the method comprises:
m/z isolating the gas phase proto membrane protein ion and/or the gas phase intact membrane protein ion by ejecting ions of m/z outside one or more pre-determined m/z ranges from the ion trap, ion guide or mass filter.

22. The method according to any preceding claim which comprises, prior to any step wherein an ion is detected, or subjected to an ion transformative process, reducing the charge of a precursor ion to produce the said ion.

23. The method according to any preceding claim which comprises determining irradiation parameters to be used in step (iii) by repeatedly performing the process and varying the energy of the laser radiation and/or the period during which the irradiation is performed until the gas phase intact membrane protein ion can be detected after step (iv).

24. The method according to any preceding claim wherein the intact membrane protein comprises two or more non-covalently associated monomers, which monomers may be the same or different.

25. The method according to any preceding claim, wherein the gas phase intact membrane protein ion comprises one or more non-covalently bound ligands.

26. The method according to claim 24 wherein the non-covalently bound ligand is selected from the group consisting of a lipid, an RNA strand, a metabolite, a drug, and a metal co-factor.

27. The method according to any preceding claim wherein the sample comprises a mass-spectrometry compatible buffer, which preferably maintains the structural integrity of the intact membrane protein in a submicron-scale membrane fragment or vesicle associated with the intact membrane protein and solvent molecules.

28. The method according to any preceding claim which is a mass spectrometric method, wherein step (v) comprises determining the m/z ratio of said gas phase intact membrane protein ion and/or an ion derived therefrom.

29. The method according to any preceding claim wherein the sample, the non-synthetic biological membrane material and/or the gas phase proto membrane protein ion is at least substantially free of detergent.


SEQUENCE LISTING (2 sequences)

SEQ ID NO: 1 - protein, 104 aa, Escherichia coli
CSTLERVVYRPDINQGNYLTANDVSKIRVGMTQQQVAYALGTPLMSDPFGTNTWFYVFRQQPGHEGVTQQTLTLTFNSSGVLTNIDNKPALSGNGGHHHHHHHH

SEQ ID NO: 2 - protein, 348 aa, unidentified
  source 1..348: note = bovine
  MOD_RES 1: note = ACETYLATION
  CARBOHYD 2: note = N-linked (Man3GlcNAc3)
  CARBOHYD 15: note = N-linked (Man3GlcNAc3)
  DISULFID 110..185: note = Intrachain disulphide bond
  LIPID 322..323: note = S-palmitoylcysteine
  MOD_RES 234: note = PHOSPHORYLATION
MNGTEGPNFYVPFSNKTGVVRSPFEAPQYYLAEPWQFSMLAAYMFLLIMLGFPINFLTLYVTVQHKKLRTPLNYILLNLAVADLFMVFGGFTTTLYTSLHGYFVFGPTGCNLEGFFATLGGEIALWSLVVLAIERYVVVCKPMSNFRFGENHAIMGVAFTWVMALACAAPPLVGWSRYIPEGMQCSCGIDYYTPHEETNNESFVIYMFVVHFIIPLIVIFFCYGQLVFTVKEAAAQQQESATTQKAEKEVTRMVIIMVIAFLICWLPYAGVAFYIFTHQGSDFGPIFMTIPAFFAKTSAVYNPVIYIMMNKQFRNCMVTTLCCGKNPLGDDEASTTVSKTETSQVAPA